(19) United States
(12) Patent Application Publication
(10) Pub. No.: US 2004/0166593 A1
(43) Pub. Date: Aug. 26, 2004

(54) Adaptive interferometric multi-analyte high-speed biosensor

(76) Inventors: David D. Nolte, Lafayette, IN (US); Fred E. Regnier, West Lafayette, IN (US); Leilei Peng, West Lafayette, IN (US)

Correspondence Address: Intellectual Property Group, Bose McKinney & Evans LLP, 2700 First Indiana Plaza, 135 North Pennsylvania Street, Indianapolis, IN, 46204, US

(21) Appl. No.: 10/726,772

(22) Filed: Dec. 3, 2003

Related U.S. Application Data
(63) Continuation-in-part of application No. 10/022,670, filed on Dec. 17, 2001, now Pat. No. 6,685,885.
(60) Provisional application No. 60/300,277, filed on Jun. 22, 2001.

(51) Int. Cl. G01N 33/543

(52) U.S. Cl. 436/518

(57) ABSTRACT

A device for identifying analytes in a biological sample, including a substrate configured to bind the analyte and a detection system to determine the presence or absence of the analyte in the biological sample.

This application is a continuation-in-part application of U.S. patent application Ser. No. 10/022,670, filed on Dec. 17, 2001, which claims the benefit of U.S. Provisional Application Ser. No. 60/300,277, filed on Jun. 22, 2001, both disclosures of which are incorporated herein by reference.

### FIELD OF THE INVENTION

The present invention generally relates to a device for detecting the presence of specific biological material in a sample, and more particularly to a laser compact disc system for detecting the presence of biological pathogens and/or analyte molecules bound to target receptors on the disc by sensing changes in the optical characteristics of a probe beam reflected from the disc caused by the pathogens and/or analytes.

### BACKGROUND OF THE INVENTION

In many chemical, biological, medical, and diagnostic applications, it is desirable to detect the presence of specific molecular structures in a sample. Many molecular structures such as cells, viruses, bacteria, toxins, peptides, DNA fragments, and antibodies are recognized by particular receptors. Biochemical technologies including gene chips, immunological chips, and DNA arrays for detecting gene expression patterns in cancer cells, exploit the interaction between these molecular structures and the receptors as described in document numbers 8-11 of the list of documents provided at the end of this specification, all of which are hereby expressly incorporated herein by reference. These technologies generally employ a stationary chip prepared to include the desired receptors (those which interact with the molecular structure under test or analyte). Since the receptor areas can be quite small, chips may be produced which test for a plurality of analytes. Ideally, many thousand binding receptors are provided for a complete assay. When the receptors are exposed to a biological sample, only a few may bind a specific protein or pathogen. Ideally, these receptor sites are identified in as short a time as possible.

One such technology for screening for a plurality of molecular structures is the so-called immunlogical compact disk, which simply includes an antibody microarray. [See documents 16-18]. Conventional fluorescence detection is employed to sense the presence in the microarray of the molecular structures under test. This approach, however, is characterized by the known deficiencies of fluorescence detection, and fails to provide a capability for performing rapid repetitive scanning.

Other approaches to immunological assays employ traditional Mach-Zender interferometers that include waveguides and grating couplers. [See documents 19-23]. However, these approaches require high levels of surface integration, and do not provide high-density, and hence high-throughput, multi-analyte capabilities.

### SUMMARY OF THE INVENTION

The present invention provides a biological, optical compact disk (“bio-optical CD”) system including a CD player for scanning biological CDs, which permit use of an interferometric detection technique to sense the presence of particular analyte in a biological sample. In one embodiment, binding receptors are deposited in the metallized pits of the CD (or grooves, depending upon the structure of the CD) using direct mechanical stamping or soft lithography. [See documents 1-7]. In another embodiment, mesas or ridges are used instead of pits. In one embodiment, the binding receptors of the mesas or ridges are deposited by microfluidic printing. [See documents 37 and 38] Since inkpad stamps can be small (on the order of a square millimeter), the chemistry of successive areas of only a square millimeter of the CD may be modified to bind different analyte. A CD may include ten thousand different “squares” of different chemistry, each including 100,000 pits prepared to bind different analyte. Accordingly, a single CD could be used to screen for 10,000 proteins in blood to provide an unambiguous flood screening.

Once a CD is prepared and exposed to a biological sample, it is scanned by the laser head of a modified CD player which detects the optical signatures (such as changes in refraction, surface shape, scattering, or absorption) of the biological structures bound to the receptors within the pits. In one example, each pit is used as a wavefront-splitting interferometer wherein the presence of a biological structure in the pit affects the characteristics of the light reflected from the pit, thereby exploiting the high sensitivity associated with interferometeric detection. For large analytes such as cells, viruses and bacteria, the interferometer of each pit is operated in a balanced condition wherein the pit depth is λ/4. For small analytes such as low-molecular weight antigens where very high sensitivity is desirable, each pit interferometer is operated in a phase-quadrature condition wherein the pit depth is λ/8. The sensitivity can be increased significantly by incorporating a homodyne detection scheme, using a sampling rate of above about 1 kHz or above about 10 kHz with a resolution bandwidth of less than 1 kHz. Since pit-to-pit scan times are less than a microsecond, one million target receptors may be assessed in one second.

These and other features of the invention will become more apparent and the invention will be better understood upon review of the following specifications and accompanying drawings.

### BRIEF DESCRIPTION OF THE DRAWINGS

FIG. 1 is a block diagram of a bio-optical CD system according to the present invention.

FIG. 2 is a top plan view of a portion of a CD.

FIGS. 3A and 3B are cross-sectional views taken substantially along lines 3A-3A and 3B-3B of FIG. 2, respectively.

FIG. 4 is a plot of the far-field diffraction of a balanced system and a system that is 20% off the balanced condition.

FIG. 5 is a plot of the far-field diffraction of a balanced system and a system operating in a condition of quadrature.

FIG. 6 is a plot of the universal response curve of interferometers.

FIG. 7 is a block diagram of the optical train of a laser according to the present invention.

FIGS. 8 and 9 are conceptual diagrams of processes for applying receptor coatings to portions of a CD.

FIG. 10 is a conceptual diagram of a method for delivering a biological sample to areas of a CD.

FIG. 11 is a top view of a CD showing a representative track.

FIG. 12A is a view of the fabrication of a stamp.

FIG. 12B is a view of the stamp of FIG. 12A stamping antibodies onto the CD of FIG. 11.

FIG. 13A is a representative view of the interaction of a probe beam with the CD of FIG. 11 wherein a signal beam is reflected from the CD.

FIG. 13B is a representative view of the interaction of a probe beam with the CD of FIG. 11 wherein a signal beam is transmitted through the CD.

FIG. 14 is a representative view of a track of a CD including FAB antibodies configured to bind a given analyte and FAB antibodies configured to not bind the given analyte.

FIG. 15 is a representative view of a process for fabricating the substrate of the CD of FIG. 14.

FIG. 16 is a representative view of fabricating a stamp to stamp antibodies onto the substrate of FIG. 15 and to complete the fabrication of the CD of FIG. 14.

FIG. 17 is a diagrammatical view of a system for detecting the presence of one or more analytes on a CD, the system including an adaptive optical element.

FIG. 18 is a representation of the interaction of a signal beam and a reference beam with the adaptive optical element of FIG. 17.

FIG. 19 is a front view of the adaptive optical element of FIG. 17.

FIG. 20 is a top view of the adaptive optical element of FIG. 17.

FIG. 21 is a diagrammatical view of a system for detecting the presence of one or more analytes on a CD, the system including an adaptive optical element.

FIG. 22 is a top view of a CD showing representative radial regions configured to bind an analyte.

FIG. 23 is a view of the CD of FIG. 22 being formed with a stamp.

### DESCRIPTION OF EXEMPLARY EMBODIMENTS OF THE INVENTION

The embodiments described below are merely exemplary and are not intended to limit the invention to the precise forms disclosed. Instead, the embodiments were selected for description to enable one of ordinary skill in the art to practice the invention.

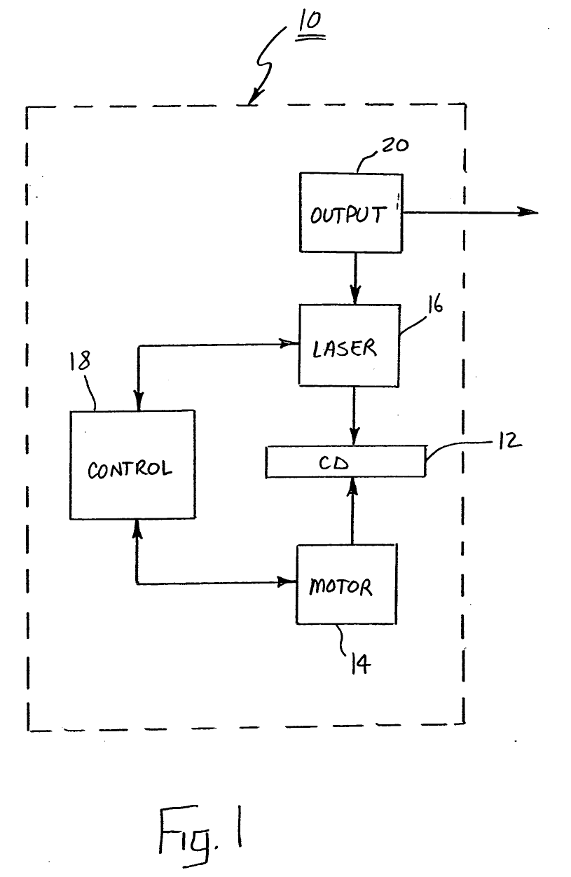

Referring now to FIG. 1, a bio-optical CD system according to the present invention generally includes a CD player 10 for scanning a removable biological CD 12. CD player 10 may be a conventional, commercial CD player modified as described herein. CD player 10 includes a motor 14, a laser 16, control electronics 18, and output electronics 20. As should be apparent to one of ordinary skill in the art, the block diagram of FIG. 1 is greatly simplified, and intended merely to suggest basic components of the well-known construction of a conventional CD player. In general, control electronics 18 control the operation of laser 16 and motor 14. Motor 14 rotates CD 12. Laser 16 obtains optical information from CD 12 as is further described below. This information is then communicated to external electronics (not shown) through output electronics 20.

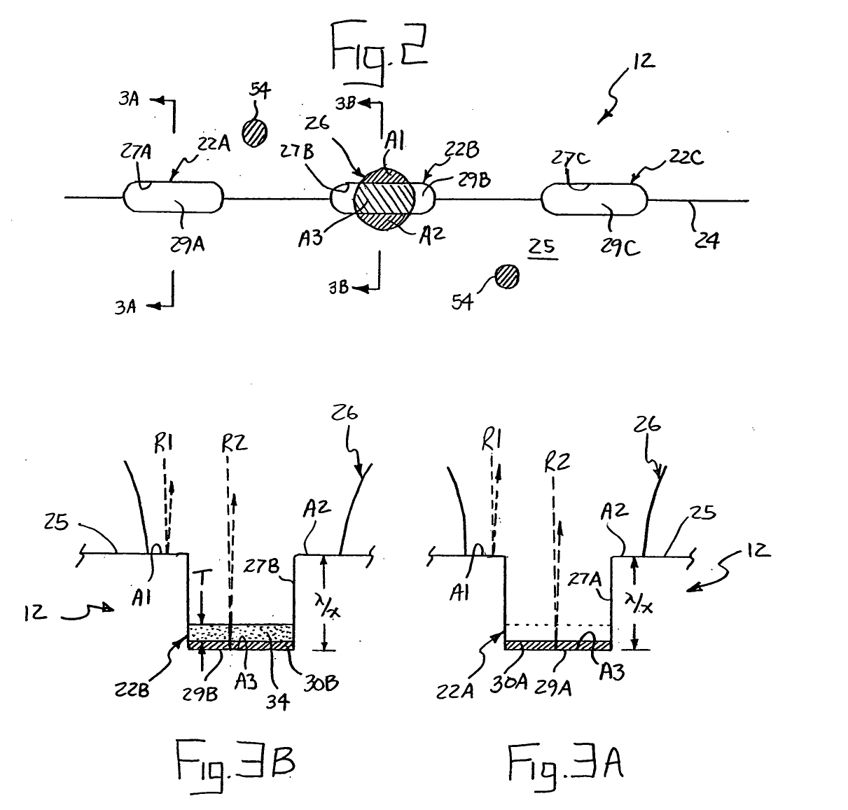

As shown in FIG. 2, CD 12 includes a substrate having a plurality of pits 22A-C (three shown) arranged on a plurality of tracks 24 (one shown). It should be understood that, while the present disclosure refers to the targets of laser 16 as “pits,” one of ordinary skill in the art could readily utilize the teachings of the invention on a CD formed with targets having different shapes, such as grooves. Moreover, as is further described below, the targets could be small plateaus, or mesas formed on the surface of the CD, or simply regions of the CD configured to bind a given analyte.

Pits 22A-C and tracks 24 are separated by flat areas of the surface of CD 12 referred to as the land 25. Each pit 22 respectively includes a sidewall 27 that extends at an angle, for example, substantially perpendicularly into the body of CD 12, and a bottom wall 29 which lies in a plane below, and substantially parallel with the plane containing land 25. According to well-established principles in the art, as CD 12 rotates, pits 22 of each track 24 move under a laser beam 26 from laser 16. After each track 24 of pits 22 is scanned, laser 16 moves laser beam 26 radially relative to the center of CD 12 to the next track 24. In this manner, laser beam 26 sequentially scans each track 24 of CD 12 until the entire area of CD 12 is scanned. It should be understood, however, that if CD 12 is formed to contain a single, spiral shaped track 24, instead of the concentric circular tracks 24 described above, laser beam 26 moves in a substantially continuous radial manner to follow the spiral of the spiral shaped track 24.

The size and position of beam 26 relative to pit 22B, for example, results in 50% of the beam area (area A1 plus area A2) reflecting off land 25, and 50% of the beam area (A3) reflecting off bottom wall 29B. Thus, CD 12 is scanned using principles of a 50/50 wavefront-splitting interferometer, as further described below.

FIG. 3A is a cross-sectional view of pit 22A under laser beam 26. A representative light ray R1 is shown reflecting off land 25 within area A1, and a ray R2 is shown reflecting off bottom wall 29A having a thin applied antibody or receptor coating 30A. Pit 22A is shown having a depth of λ/x. Pits of conventional CDs have a depth of λ/4. On double pass (on reflection), this depth imparts a π phase shift to the light incident in pit 22A relative to the light incident on areas A1 and A2 of land 25. In other words, because the distance traveled by ray R2 is approximately λ/2 times greater than the distance traveled by ray R1 (λ/4 down pit 22A plus λ/4 up pit 22A ignoring the thickness of coating 30A), the reflected ray R2 appears phase shifted by one-half of one wavelength. As explained with reference to FIG. 2, the intensity of light incident on pit 22A (within area A3) is balanced by the intensity of light on land 25 (within areas A1 and A2). The equal reflected amplitudes and the π phase difference between the light reflected from pit 22A and land 25 cause cancellation of the far-field diffracted intensity along the optic axis. The presence of pit 22A is therefore detected as an intensity drop-out as laser 16 scans over the surface of CD 12. This drop out is due to the destructive interference of the light from land 25 and pit 22A. Splitting the amplitude between pit 22A and land 25 creates the 50/50 wavefront splitting interferometer. [See document 24].

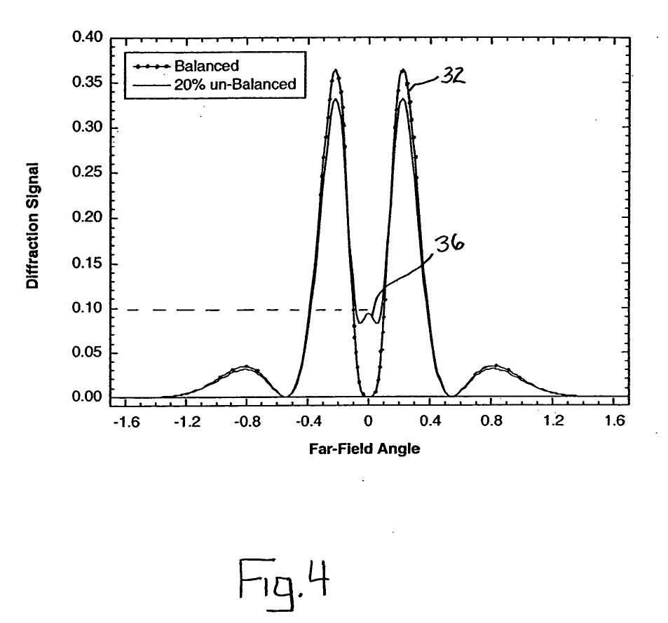

The far-field diffraction of pit 22A is shown as signal 32 in FIG. 4 for the balanced condition with a π phase difference between pit 22A and land 25. The intensity is cancelled by destructive interference along the optic axis. At finite angles, the intensity appears as diffraction orders. During immunological assays, it is common to use antibodies to bind large pathogens such as cells and bacteria. These analytes are large, comprising a large fraction of the wavelength of light. For instance, the bacterium E coli has a width of approximately 0.1 microns and a length of about 1 micron. While this bacterium is small enough to fit into a pit 22A-C, it is large enough to produce a large phase change from the pit 22A-C upon binding.

In this situation of a large analyte, the interferometer is best operated in the balanced condition described above. The presence of the analyte is detected directly as a removal of the perfect destructive interference that occurs in the absence of the bound pathogen as described below. It should also be understood that to improve detection sensitivity, it is possible to attach tags to bound analytes that can turn small analytes into effective large analytes. Conversely, sandwich structures can be used to bind additional antibodies to the bound analytes that can improve the responsivity of the detection.

When the balanced phase condition is removed, only partial destructive interference occurs. Referring to FIG. 3B, pit 22B is shown under beam 26. The structure of pit 22B of FIG. 3B is identical to that of pit 22A of FIG. 3A, except that receptor coating 30B has attracted a molecular structure 34 from the biological sample under test. Molecular structure 34 is shown as having a thickness T. As light ray R2 travels through thickness T of structure 34, ray 32 acquires additional phase because of the refractive index of structure 34. Specifically, since pit 22B has a depth of λ/4 (like pit 22A of FIG. 3A), and structure 34 has a thickness T, ray R2 travels in a manner that yields a phase shift of some percentage of λ/2. Assuming T is sufficiently large to result in a phase difference of 0.8*(λ/2), a diffraction signal 36 results as shown in FIG. 4. Signal 36 is approximately 10% (relative to 100% for light incident entirely on land 25) greater at a far-field diffraction angle of zero. Accordingly, one embodiment of a system of the present invention may detect the presence of particular molecular structures within a biological sample by detecting changes in diffraction signal as described above.

It should be apparent that since the system detects changes in amplitude of light from one area (A3) relative to light reflected from another area (A1 plus A2), land 25 could be coated with receptor coating (not shown) instead of bottom walls 29A-C of pits 22A-C to yield the same result. In such an embodiment, molecular structure 34 binds to the coating (not shown) on land 25 adjacent pit 22A-C, thereby affecting the phase of representative light ray R1. This difference manifests itself as a change in the diffraction signal in the manner described above.

As indicated above, in an alternate embodiment of the invention, mesas are used instead of pits 22A-C. According to this embodiment, flat plateaus or mesas are formed at spaced intervals along tracks 24. Such mesas may be formed using conventional etching techniques, or more preferably, using deposition techniques associated with metalization. All of the above teachings apply in principle to a CD 12 having mesas instead of pits 22A-C. More specifically, it is conceptually irrelevant whether rays R1 and R2 acquire phase changes due to the increased travel of ray R2 into a depression or pit, or due to the reduced travel of ray R2 as it is reflected off the upper wall of a raised plateau or mesa. It is the difference between the travel path of ray R2 and that of ray R1 that creates the desired result.

Alternatively, because some cells and bacteria are comparable in size to the wavelength of light, it should also be possible to detect them directly on a flat surface uniformly coated with binding receptors, such as antibodies or proteins, rather than bound in or around pits 22A-C. This has the distinct advantage that no pit (or mesa) fabrication is needed, and the targets can be patterned into strips that form diffraction gratings (see Ref. 27&28). Alternatively, it is often adequate in an immunological assay simply to measure the area density of bacteria. As laser 16 scans over the bacterium, the phase of the reflected light changes relative to land 25 surrounding the bacterium. This causes partial destructive interference that is detected as dips in the reflected intensity.

The contrast between the balanced (empty) pit and the binding pit can be large. However, high signal-to-noise-ratio (SNR) requires high intensities, which is not the case when the interferometer is balanced. Accordingly, another embodiment of the present invention employs homodyne detection that uses pit depths resulting in amplitudes from the pit and land in a condition of phase-quadrature as described below.

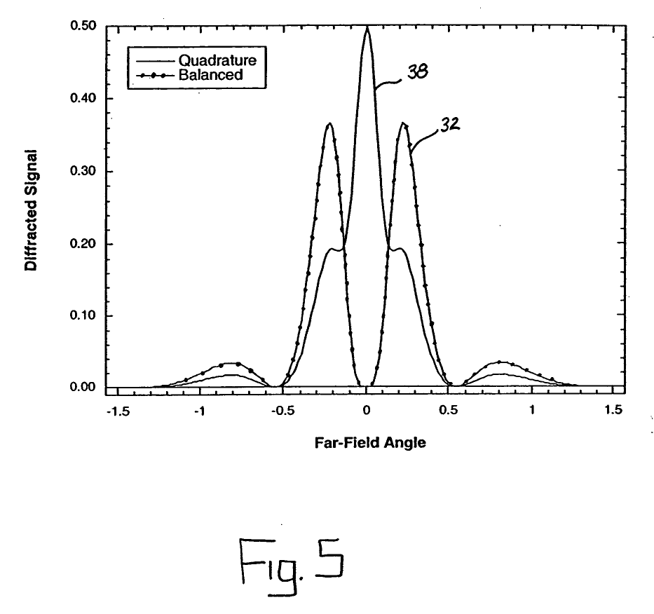

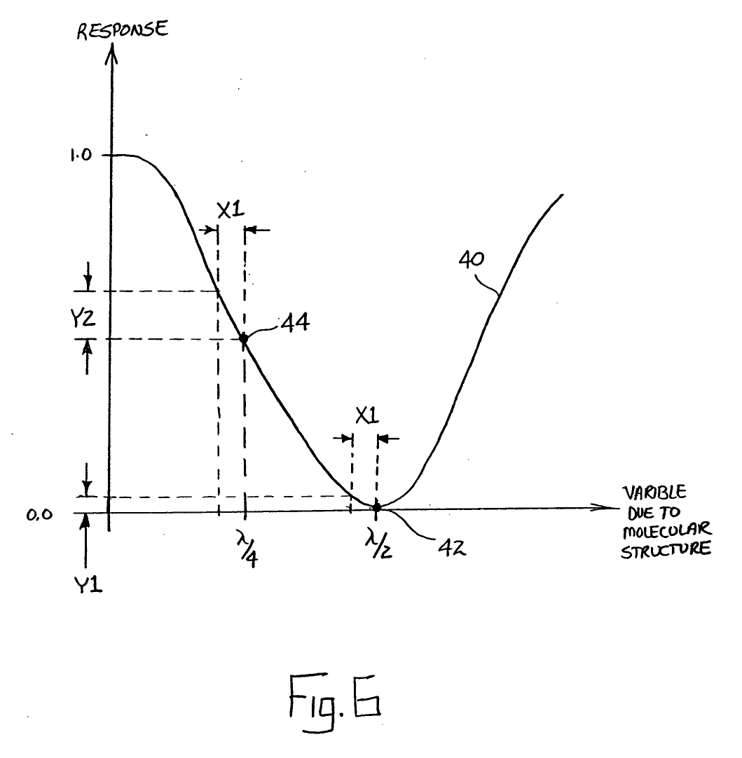

Phase-quadrature is attained when the two amplitudes (the light intensity reflected from pit 22A, for example, and the light intensity reflected from areas A1 and A2 of land 25 surrounding pit 22A) differ by a phase of λ/2. This condition thus requires a pit depth of λ/8. It is well-known that the quadrature condition yields maximum linear signal detection in an interferometer. [See document 25]. The far-field diffraction of a pit in the condition of quadrature is shown as signal 38 in FIG. 5. In this condition, very small changes in the relative phase of the pit and land cause relatively large changes in the intensity along the optic axis. For example, a phase change of only 0.05*(λ/2) produces the same magnitude change in the diffracted signal as the relatively large phase change of 0.2*(λ/2) which resulted in signal 36 of FIG. 4. Accordingly, the condition of quadrature provides much higher sensitivity for detection of small bound molecular structures.

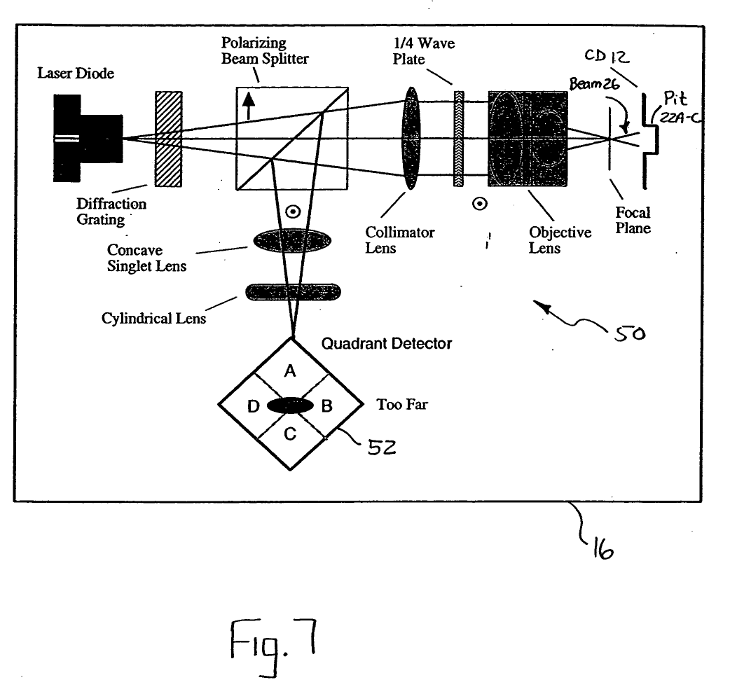

FIG. 6 further depicts the differences in response characteristics of the two modes of operation described above. Curve 40 represents the universal response curve of all interferometers. Optical CD systems operating in a balanced condition as described above function at and around the point 42 of curve 40 corresponding to λ/2 on the x-axis of the figure. As should be apparent from the drawing, changes in the measured response (for example, light reflection) resulting from changes due to the presence of the sensed molecular structure (for example, the distance traveled by ray R2 of FIGS. 3A, 3B), are relatively small when operating about point 42 because of the low slope of curve 40. Specifically, a change of X1 along the x-axis of FIG. 6 results in a change in response of Y1.

When operating in the condition of quadrature, on the other hand, a CD system according to the present invention operates at and around the point 44 of curve 40 corresponding to λ/4 on the x-axis of FIG. 6. Clearly, this portion of curve 40 yields a more responsive system because of its increased slope. As shown, the same change of X1 that resulted in a change in response of Y1 relative to point 42 yields a much greater change in response of Y2 relative to point 44.

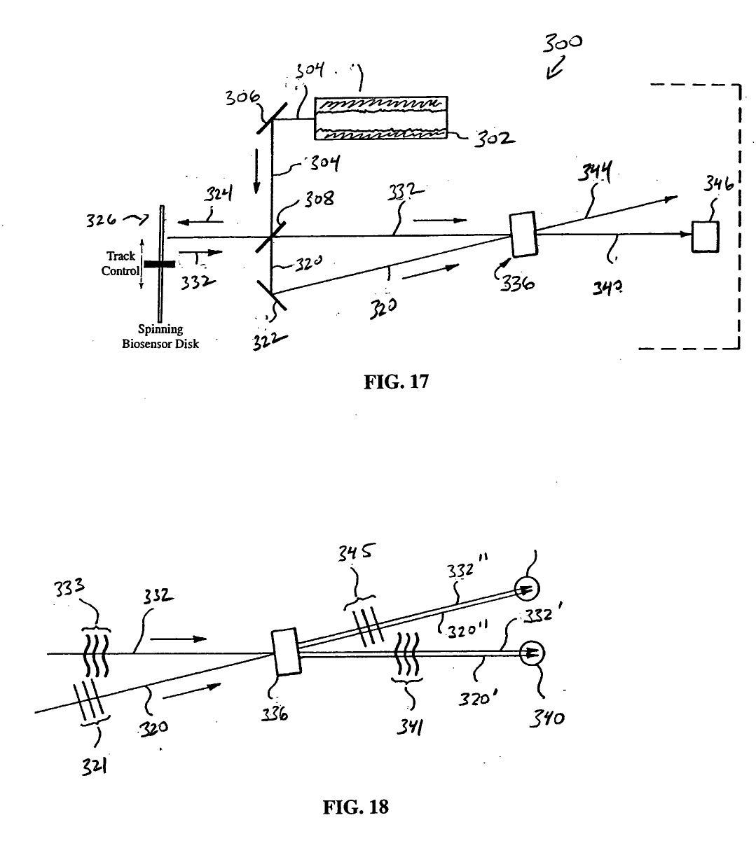

As should be apparent from the foregoing, regardless of the depth of pits 22A-C, or even whether pits are used at all, the presence or absence of analytes creates a phase modulated signal, which conveys the screening information. If one desires to maintain a quadrature condition and its associated increased sensitivity, the technology described in U.S. Pat. No. 5,900,935, which is incorporated herein by reference, may be adapted. Instead of a phase modulated signal from an ultrasound source, the present invention so adapted provides a phase modulated signal from analytes as described above. FIGS. 17-21 demonstrate exemplary embodiments of the technology described in U.S. Pat. No. 5,900,935 adapted to provide a phase modulated signal.

It is possible to derive equations describing the fundamental SNR for detection in quadrature as a homodyne detection process. The intensity along the optic axis of the detection system when it is in quadrature is given by

$$I = ( {I}_{1} + {I}_{2} ) ( 1 + m cos ( \frac{π}{4} + δ ) ) \qquad ( 1 )$$

where I₁ and I₂ are the intensities reflected from land 25 and a particular pit 22A-C. The phase shift of the light reflected from pit 22A-C is

$$δ = \frac{4 π}{λ} Δ {nd}_{An} \qquad ( 2 )$$

where Δn is the change in refractive index cause by the bound molecular structure, and d_{An} is the thickness of the bound molecular structure. The contrast index m is given by

$$m = \frac{2 \sqrt{{I}_{1} {I}_{2}}}{{I}_{1} + {I}_{2}} \qquad ( 3 )$$

For ideal operation, P₁=P₂, P+P₂, and m =1.

For small phase excursions, the signal detected from Eq. 1 becomes

$$S = \frac{P}{\sqrt{2 h v}} m \frac{4 π}{λ} Δ {nd}_{An} \qquad ( 4 )$$

in terms of the total detected powers P and where hv is the photon energy. There are three sources of noise in this detection system: 1) shot noise of the light from beam 26; 2) binding statistics of the antibodies; and 3) bonding statistics of the bound analyte. The shot noise is given by

$${N}_{shot} = \sqrt{\frac{P}{hv BW}} \qquad ( 5 )$$

where BW is the detection bandwidth of the detection system. The noise from the fluctuations in the bound antibody is given by (assuming random statistics)

$${N}_{Ab} = \frac{P}{h v} m \frac{4 π}{λ} Δ {n}_{Ab} \sqrt{{M}_{Ab}} {d}_{Ab}^{0} \qquad ( 6 )$$

and for the bound analyte is

$${N}_{An} = \frac{P}{hvBW} m \frac{4 π}{λ} Δ {n}_{An} \sqrt{{M}_{An}} {d}_{An}^{0} \qquad ( 7 )$$

where M_{Ab} and M_{An} are the number of bound antibody and analyte molecules, and d_{An}⁰ and d_{Ab} ⁰ are the effective thicknesses of a single bound molecule given by

$${Ad}_{An}^{0} = {V}_{An}^{0} \qquad ( 8 )$$

where A is the area of pit 22A-C and V_{An}⁰ is the molecular volume.

The smallest number of analyte molecules that can be detected for a SNR equal to unity, assuming the analyte fluctuation noise equals the shot noise, is given by the NEM (noise-equivalent molecules)

$$NEM = \frac{hv BW}{P} {( \frac{λ}{4 π Δ {n}_{An} {d}_{An}^{0}} )}^{2} \qquad ( 9 )$$

A detected power of 1 milliwatt and a detection bandwidth of 1 Hz, assuming Δn=0.1 and d_{An}⁰=0.01 picometer, yields a one-molecule sensitivity of

NEM≈1 (10)

This achieves sensitivity for single molecule detection with a SNR of unity. To achieve a SNR of 100:1 would require 10,000 bound molecular structures.

An alternative (and useful) way of looking at noise is to calculate the noise-equivalent power (NEP) of the system. This is defined as the power needed for the shot noise contribution to equal the other noise contributions to the total noise. Assuming that the antibody layer thickness fluctuations dominate the noise of the system, the NEP is obtained by equating Eq. 5 with Eq. 6. The resulting NEP is

$$NEP = \frac{h v BW}{{( 4 πΔ {n}_{Ab} )}^{2} {M}_{Ab}} {( \frac{λ}{{d}_{Ab}^{0}} )}^{2} \qquad ( 11 )$$

If an antibody layer thickness of 0.01 pm and a refractive index change of 0.1 are assumed, the resulting NEP is 1 milliwatts· molecules. If there are 10⁵ bound antibodies in a pit (or within the radius of the probe laser), then the power at which the shot noise equals the noise from the fluctuating antibody layer thickness is only

NEP=10 n Watts/Hz (12)

Accordingly, probe spot powers greater than 10 nW will cause the noise to be dominated by the fluctuating antibody layer thickness rather than by the shot noise. The NEP is therefore an estimate of the required power of laser 16. In this case, the power is extremely small, avoiding severe heating.

FIG. 7 depicts an optical train 50 included within laser 16 of FIG. 1 for detecting bound analytes. Optical train 50 is identical to conventional optical trains currently used in commercial CD-ROM disks. Vertical tracking is accomplished “on-the-fly” using a four-quadrant detector 52 and a servo-controlled voice coil to maintain focus on the plane of spinning CD 12. Likewise, lateral tracking uses two satellite laser spots 54 (FIG. 2) with a servo-controlled voice coil to keep probe laser spot 26 on track 24. This approach uses the well-developed tracking systems that have already been efficiently engineered for conventional CD players. The high-speed real-time tracking capabilities of the servo-control systems allows CD 12 to spin at a rotation of 223 rpm and a linear velocity at the rim of 1.4 m/sec. The sampling rate is 4 Msamp/sec, representing very high throughput for an immunological assay. The ability to encode identification information directly onto CD 12 using conventional CD coding also makes the use of the CD technology particularly attractive, as patented in U.S. Pat. No. 6,110,748.

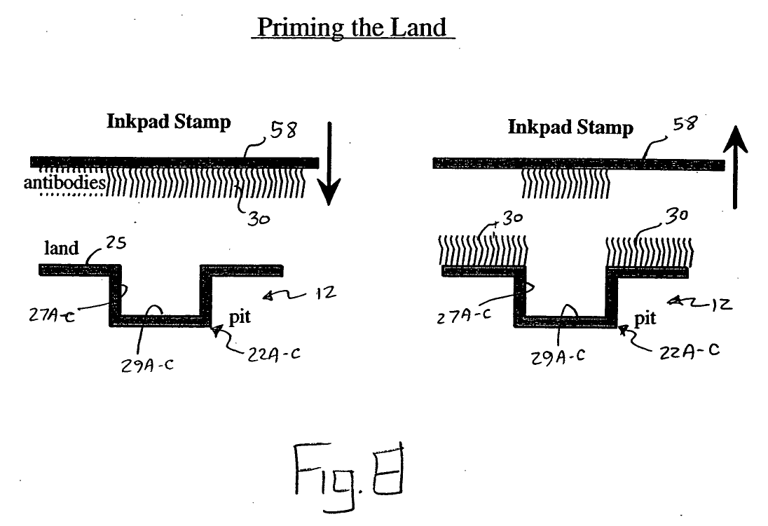

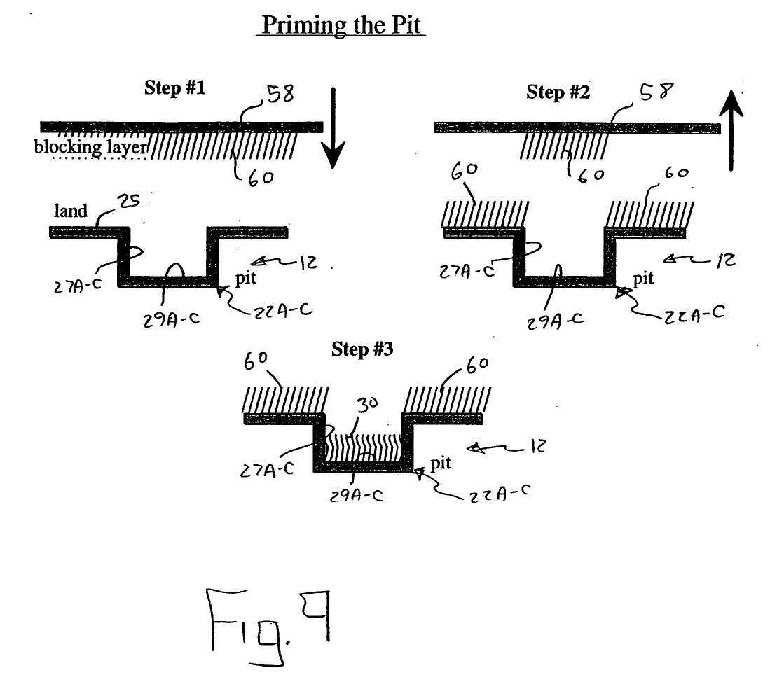

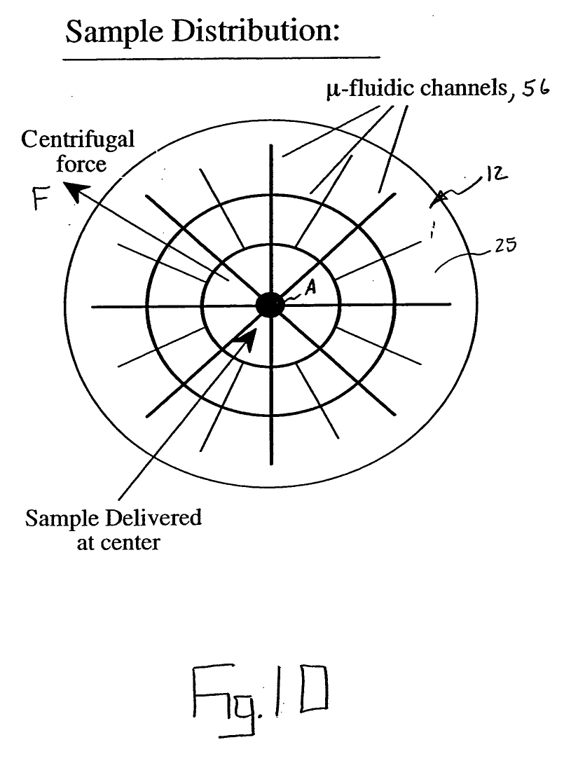

CD 12 can be charged using novel inkpad stamp technology [see documents 1-7] shown in FIGS. 8 and 9. Either land 25 or pits 22A-C can be primed with antibody layer 30. To prime land 25, the antibodies coated on the inkpad 58 attach only on land 25 that is in contact with pads 22A-C, as shown in FIG. 8. Analytes bound on land 25 are equally capable of changing the far-field diffraction as analytes bound to the pits 22A-C. Of course, as described below, the antibodies may be coated (receptor coating 30) on bottom wall 29A-C of pits 22A-C.

Referring now to FIG. 9, to prime antibodies in pits 22A-C, first a blocking layer 60 can be applied to land 25 that prevents the adhesion of antibodies 30. Later, the area is flooded with antibodies 30 that only attach in exposed pits 22A-C. Blocking layer 60 can later be removed to improve the sensitivity of the optical detection (by removing the contribution to the total noise of the detection system of the fluctuations of the thickness of blocking layer 60).

The delivery of biological samples containing analytes to the primed areas of bio-CD 12 (i.e., pits 22A-C, land 25, or simply a flat surface of CD 12) can be accomplished using microfluidic channels 56 fabricated in CD 12, as shown in FIG. 10. Microfluidic channels 56 can plumb to all pits 22A-C. Alternatively, the biological sample can flow over land 25. The advantages in spinning CD 12 is the use of centrifugal force F to pull the fluid biological sample from the delivery area near the central axis A over the entire surface of CD 12 as an apparent centrifuge, as in U.S. Pat. No. 6,063,589. Similarly, capillary forces can be used to move the fluid through microchannels 56. This technique of biological sample distribution can use micro-fluidic channels 56 that are lithographically defined at the same time CD pits 22A-C are defined.

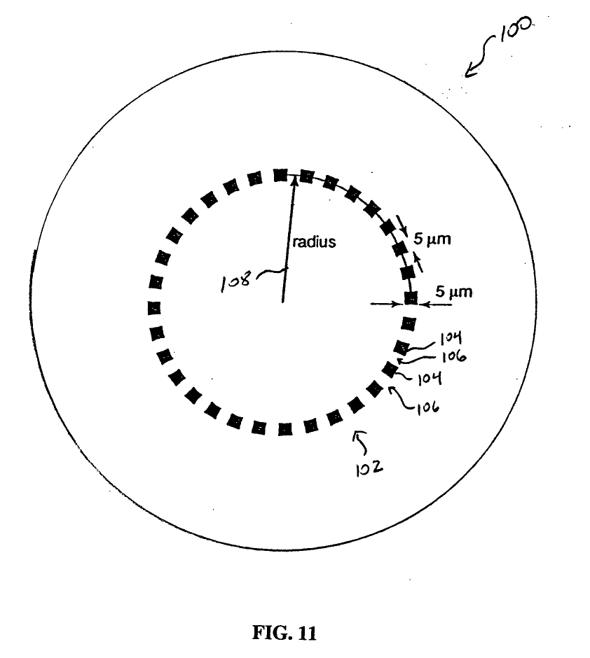

Referring to FIGS. 11-13, another embodiment of a CD for use with the present invention is described, CD 100. CD 100 includes concentric tracks 102 (one shown) of regions or targets 104 configured to bind a given analyte and reference blanks 106 configured to not bind the given analyte which targets 104 are configured to bind. Regions 104 and blanks 106 are arranged in a repeating pattern, such as alternating between regions 104 and blanks 106. In one example, a typical region 104 or blank 106 has an extent of approximately about 5 microns to about 10 microns, such that a track 102 having a radius 108, of about 1 centimeter includes between about 5,000 to about 10,000 regions 104 and blanks 106. Further, assuming that a CD can hold about 10,000 tracks with a spacing between tracks of about 5 microns, CD 100 could have about 100,000,000 regions 104 and blanks 106.

In another embodiment, regions 104 are radial spokes on CD 100, similar to CD 200′ described below. The radial spokes are formed on CD 100 by microfluidic printing as described herein or inkjet technology as described herein.

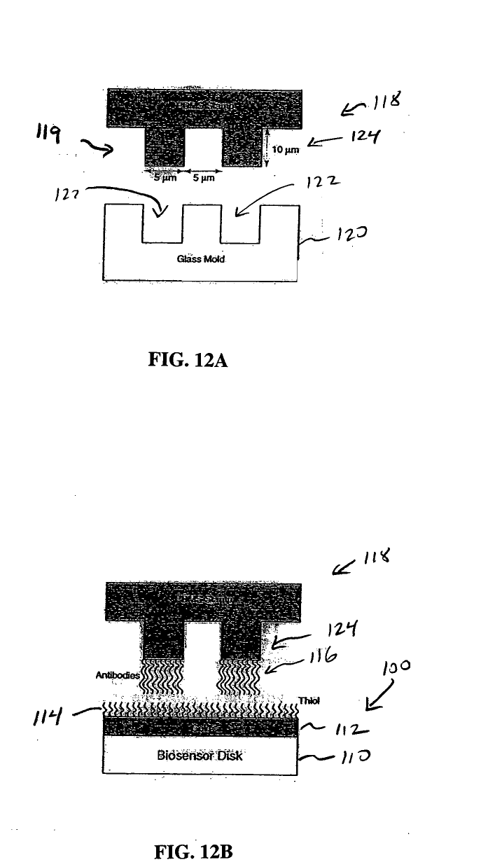

CD 100, in one embodiment, is fabricated as follows. Referring to FIGS. 12A and 12B, CD 100 is fabricated by soft-lithography or ink-pad stamping. [See documents 1, 3, and 6]. CD 100 includes a glass substrate 110 (a silica optical flat) coated with a layer 112 of gold which is functionalized with thiol groups 114 configured to bind an analyzer molecule 116, such as antibodies or cDNA. In one example, the thickness of gold layer 112 is about 50 nanometers to about 100 nanometers thick. Next analyzer molecules 116, antibodies or cDNA, are applied to the thiol groups 114 in select regions to form regions 104. The areas where analyzer molecules 116 are not applied are designated as blanks 106.

In one embodiment, analyzer molecules 116 are applied to thiol groups 114 with a stamp 118. Stamp 118 is formed from a glass mold 120 which is fabricated using conventional photolithography and ion milling to create recesses 122 in the locations corresponding to regions 104 on CD 100. Stamp 118, in one example, is made of polydimethylsiloxane (PDMS). Stamp 118, in the illustrated embodiment, is inked with monoclonal antibodies or cDNA 116 which attach to raised portions 124 of stamp 118 corresponding to recesses 122 in mold 120. The inked antibodies or cDNA 116 are subsequently stamped onto CD 100 to form regions 104 and blanks 106. It should be appreciated that stamp 118 includes tracks not in FIG. 119 (one shown) which correspond to tracks 102 of CD 100.

In order to detect multiple analytes with CD 100, a track 102 is created for each of the analytes to be detected. As stated above, in one example up to about 10,000 tracks may be created on CD 100. Each track 102 requires an analyzer molecule 116, such as an antibody, protein, or cDNA, configured to bind the analyte that is to be associated with the respective track 102. In one exemplary method wherein CD 100 is configured to bind multiple analytes, stamp 118 is created with a direct-write technique using a microfluid pen. The microfluid pen is filled with a given analyzer molecule 116, such as an antibody, and an associated track 119 of stamp 118 is rotated underneath the stationary microfluid pen such that analyzer molecule 116 inside of the pen is applied to raised portions 124 of track 119. The microfluid pen is then flushed and filled with a second analyzer molecule 116. The pen containing the second analyzer molecule 116 is positioned over a second track 119 of stamp 118 and the second analyzer molecule 116 is applied to the second track 119 as described above. This process is repeated until all of the tracks 119 which are to be associated with one of a plurality of given analytes are applied. Stamp 118 is then stamped onto CD 100 to form CD 100. The resultant CD 100 may be used with the detection system described above and detection systems 300, 400 discussed below.

In another embodiment, CD 100 is fabricated using inkjet technology. Substrate 110 of CD 100 is coated with a layer 112 of gold which is functionalized with thiol groups 114 configured to bind an analyzer molecule 116, such as antibodies, proteins, or cDNA. In one example, the thickness of gold layer 112 is about 50 nanometers to about 100 nanometers thick. Next analyzer molecules 116, are applied to the thiol groups 114 in select regions to form regions 104 through inkjet techniques. The areas where analyzer molecules 116 are not applied are designated as blanks 106.

In one exemplary method analyzer molecules 116 are deposited on CD 100 with inkjet technology. A reservoir associated with an inkjet head is filled with a given analyzer molecule 116, such as an antibody, protein, or cDNA, and an associated track 102 of CD 100 is rotated underneath the inkjet head such that analyzer molecule 116 associated with the inkjet head is applied to portions of CD 100 corresponding to regions 104 of track 102. As is understood in the inkjet art, the inkjet head is controlled to apply analyzer molecules 116 to the selected regions 104 of track 102 and to not apply analyzer molecules 116 to regions 106 of track 102. In alternative embodiments, CD 100 is held stationary and the inkjet head is moved across the surface of CD 100.

In one exemplary method, multiple analyzer molecules are bound to respective tracks 102 of CD 100 with inkjet technology by filling the reservoir associated with the inkjet head with a first analyzer molecule, creating the first track 102 on CD 100, flushing the inkjet head and associated reservoir, filling the associated reservoir with a second analyzer molecule, and creating a second track 102 on CD 100. This process may be repeated to create further tracks 102 on CD 100.

In another exemplary method, multiple analyzer molecules are bound to respective tracks 102 of CD 100 with inkjet technology by providing multiple reservoirs and multiple associated inkjet heads, filling each reservoir with a respective analyzer molecule, simultaneously applying the analyzer molecules to CD 100 to create respective tracks 102 on CD 100. If desired the multiple inkjet heads and associated reservoirs may be flushed and filled with further analyzer molecules to form additional tracks on CD 100.

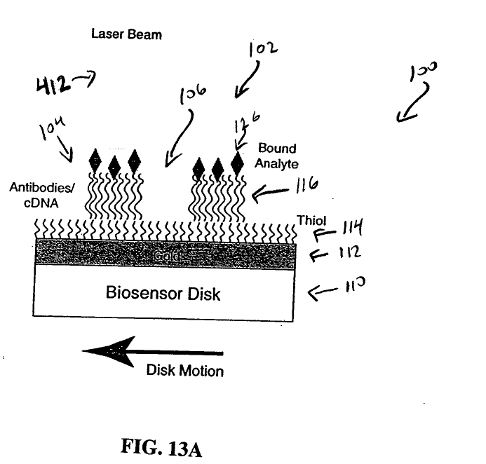

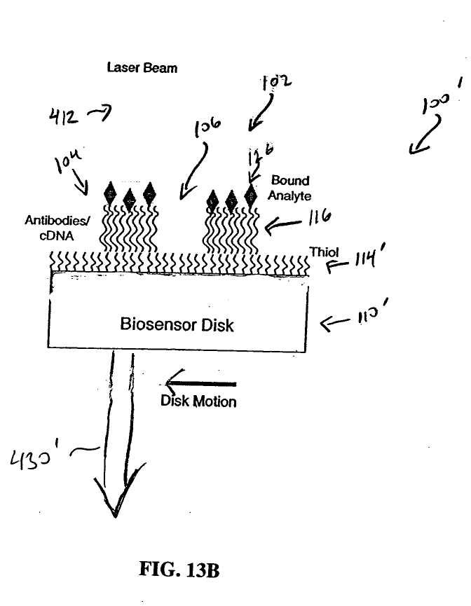

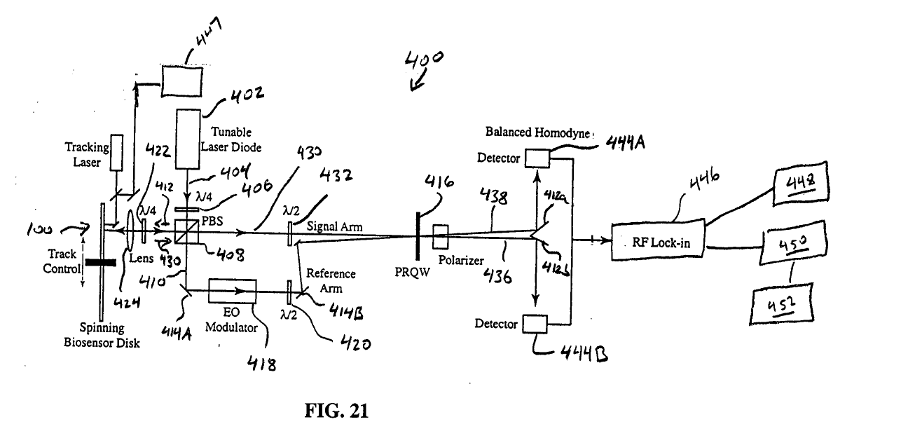

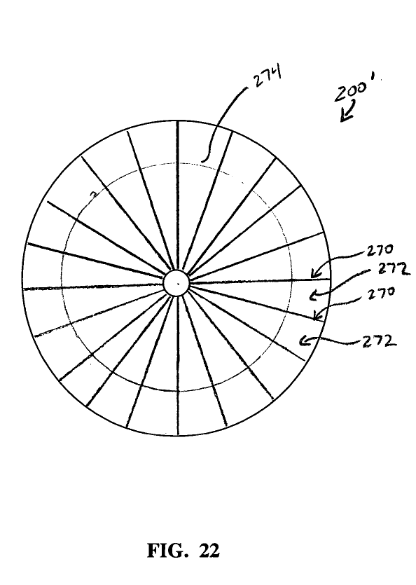

Referring to FIG. 13A, a probe beam from a laser, such as probe beam 304 (FIG. 17) or 412 (FIG. 21) described below, is incident on CD 100, is altered by the characteristics of CD 100 including the presence or absence of bound analytes 126, the presence or absence of analyzer molecules 116, and the presence of thiols 114, and is reflected by gold layer 112. The reflected beam is detected by the detection system described above and detection systems 300, 400 discussed below. In one example, the probe laser sweeps across targets 104 or reference blanks 106 along with the region surrounding targets 104 or reference blanks 106 with a duty cycle of approximately 50 percent. Further, the probe laser continues to scan over track 102 until a determination is made regarding the presence or absence of the analyte configured to bind to targets 104 of track 102. In one example, CD 100 includes microfluid channels, similar to CD 12, to deliver a biological sample to targets 104.

Referring to FIG. 13B, a CD 100′ is shown along with a probe beam from a laser, such as probe beam 304 (FIG. 17) or 412 (FIG. 21) described below. CD 100′ is generally similar to CD 100 except that CD 100′ is configured to produce a transmitted signal beam 332′ or 430′ compared to a reflected signal beam 332 or 430. In the illustrated embodiment, CD 100′ includes a substrates 10′ configured to permit the transmission of optical energy and a layer of silane 114′. Layer 114 is configured to bind analyzer molecules 116 which as explained herein are configured to bind analytes 126.

Illustratively shown in FIG. 13B, probe beam 412 is incident on CD 100′, is transmitted through CD 100′ to produce signal beam 430′ and is altered by the characteristics of CD 100′ including the presence or absence of bound analytes 126, the presence or absence of analyzer molecules 116, and the saline layer 114′, and substrate 110′. In one example, substrate 110′ is a glass substrate. Transmitted signal beam 430′ is detected by the detection system described above and detection systems 300, 400 discussed below. In one example, the probe laser sweeps across targets 104 or reference blanks 106 along with the region surrounding targets 104 or reference blanks 106 with a duty cycle of approximately 50 percent. Further, the probe laser continues to scan over track 102 until a determination is made regarding the presence or absence of the analyte configured to bind to targets 104 of track 102. In one example, CD 100′ includes microfluid channels, similar to CD 12, to deliver a biological sample to targets 104. It should be understood that CD 12, CD 200, and CD 200′ may also be configured to produce a transmitted signal beam as opposed to a reflected signal beam.

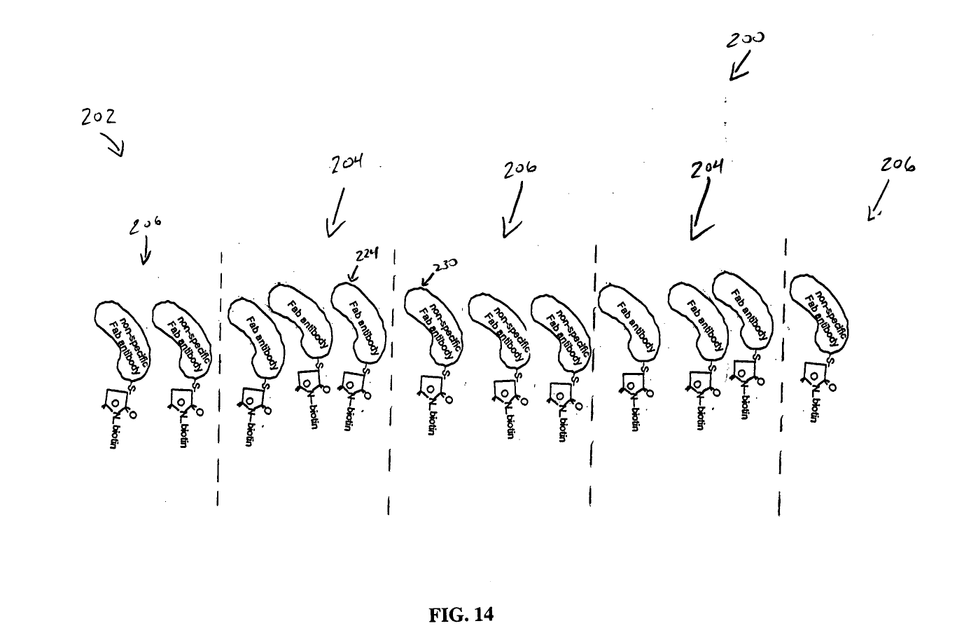

Referring to FIGS. 14-16, another embodiment of a CD for use with the present invention is described, CD 200. CD 200 includes tracks 202 (one shown) of alternating specific analyte binding regions or targets 204 configured to bind a given analyte and non-specific analyte binding regions 206 arranged in a repeating pattern, such as alternating between regions or targets 204 and regions 206. In the illustrated embodiment, targets 204 are coated with an analyzer molecule 224, such as a FAB (fragment antibody) antibody or a protein, that is configured to bind a specific analyte while regions 206 are coated with a blocking material which is configured not to bind the analyte that target 204 is configured to bind, such as a non-specific molecule. Exemplary non-specific molecules include a FAB antibody 230 or a protein.

In one example, tracks 202 are concentric circular tracks. In another example, tracks 202 are formed as one or more spiral tracks. In yet another example, multiple tracks 202 are positioned on a single concentric, circular track or a single spiral track.

It should be appreciated that CD 200 does not have regions of varying heights such as pits and lands or mesas and lands like CD 12. On the contrary, CD 200 is generally uniform. Since CD 200 is generally uniform (in the absence of analyzer molecules binding an analyte) a periodic or phase modulated signal is not detected using detection systems 300, 400 described below until an analyte configured to be bound by an analyzer molecule 224 of a given track 202 is introduced. Once the analyte is introduced, the analyte binds to the analyzer molecules 224 of targets 204 of given track 202. The binding of the analyte in target regions 204 and not in regions 206 causes the generation of a phase modulated signal from track 202 when CD 200 is spun and track 202 is being monitored by one of system 300, 400. The phase modulated signal is created by the successive probing of targets 204 and regions 206 while CD 200 is spinning.

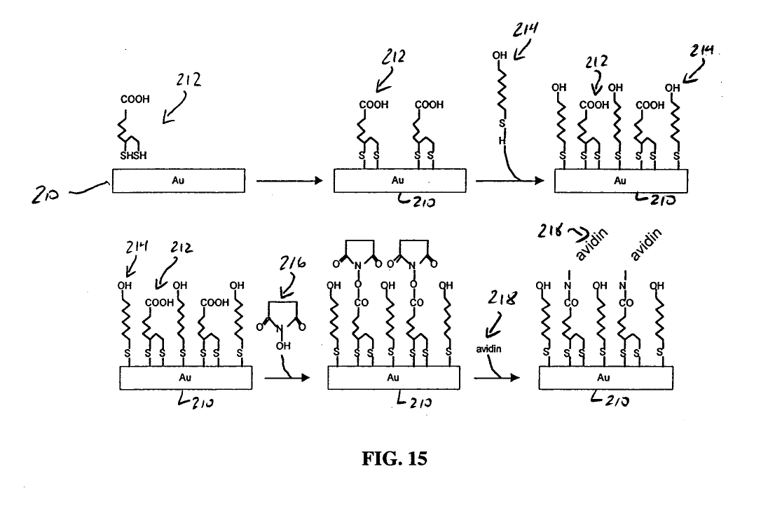

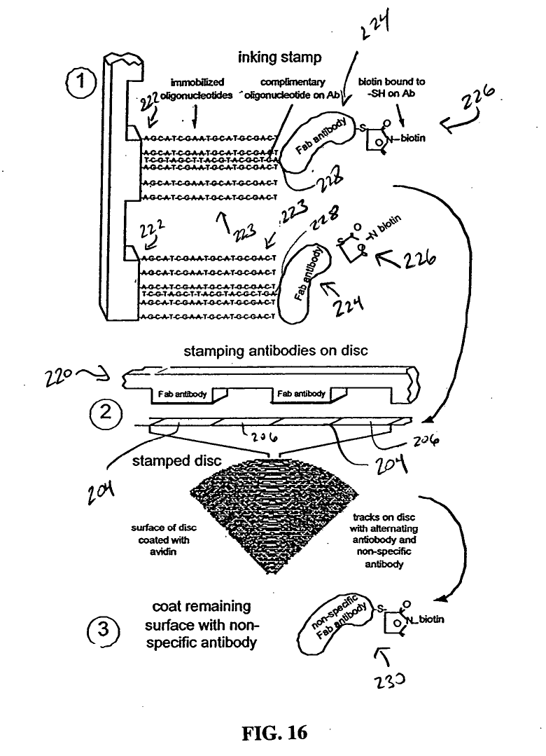

Referring to FIGS. 15 and 16, a method of fabricating CD 200 is shown. FIG. 15 illustrates the preparation of the CD surface. FIG. 16 illustrates application of specific regions 204 and non-specific regions 206 to the surface of CD 200. Referring to FIG. 15, CD 200 includes a glass substrate (not shown) and a gold layer 210. A first thiol molecule 212 is bonded to gold layer 210 followed by the binding of a second thiol molecule 214 to gold layer 210. First thiol molecule 212 and second thiol molecule 214 are bound to gold layer 210 in a generally alternating fashion. Next, molecule 216 is bound to the carboxylic acid group of first thiol molecule 212. Finally, avidin 218 is bound to molecule 216. In one embodiment, the entire surface of CD 200 is coated with avidin 218. In another embodiment, specific areas of CD 200 which correspond to tracks 202 of CD 200 are coated with avidin 218.

Referring to FIG. 16, a stamp 220 is prepared which is used to stamp regions or targets 204 onto the avidin coated CD. As shown in FIG. 16, in specific regions 222 of stamp 220 immobilized nucleotide oligos 223 are deposited in arrays. Each FAB antibody 224 is attached to a biotin 226 and to a complementary oligo strand 228. Complementary oligo strands 228 are selected to match the immobilized nucleotide oligos for a particular track such that when a given FAB antibody is washed over the surface of stamp 220, the complementary oligo strand 228 will only match or align with the matching immobilized nucleotide oligos 223 resulting in the given FAB antibody 224 being selectively positioned in areas of stamp 220 which ultimately coincide with regions or targets 204 of track 202 of CD 200.

Once all of the specific FAB antibodies 224 have been properly positioned on stamp 220, stamp 220 is positioned such that specific FAB antibodies 224 are stamped onto the avidin coated CD. The biotin 226 attached to the specific FAB antibodies 224 binds to the avidin 218 on CD 200 such that specific FAB antibodies 224 are positioned on CD 200 to form regions or targets 204. After CD 200 has been stamped, CD 200 is coated with non-specific FAB antibody 230 which binds to avidin 218 in the remaining areas of CD 200 forming regions 206. After which, CD 200 includes tracks 202 having targets 204 and regions 206, such as the partial track 202 shown in FIG. 14. In one example, CD 200 includes microfluid channels, similar to CD 12, to deliver a biological sample to targets 204.

Referring to FIG. 22, CD 200′ is shown. CD 200′ is generally similar to CD 200 and is configured to include radial regions 270 configured to bind a given analyte and radial regions 272 configured not to bind a given analyte, the regions 270 and regions 272 being arranged in a repeating pattern. By spinning CD 200′ and scanning CD 200′ along a given circular path, such as path 274 with one of detection systems 300, 400 the presence or absence of the given analyte may be detected.

To detect the presence of multiple analytes a sector of CD 200′ is configured to include regions 270 configured to bind a specific analyte. In one example, a first sector of CD 200′ corresponding to about one-third of the area of CD 200′ is configured to bind a first analyte, a second sector of CD 200′ corresponding to about one-third of the area of CD 200′ is configured to bind a second analyte, and a third sector of CD 200′ corresponding to about one-third of the area of CD 200′ is configured to bind a third analyte. When CD 200′ is configured to detect the presence of multiple analytes, CD 200′ includes a synchronization pattern to provide the detection system with a reference point on CD 200′.

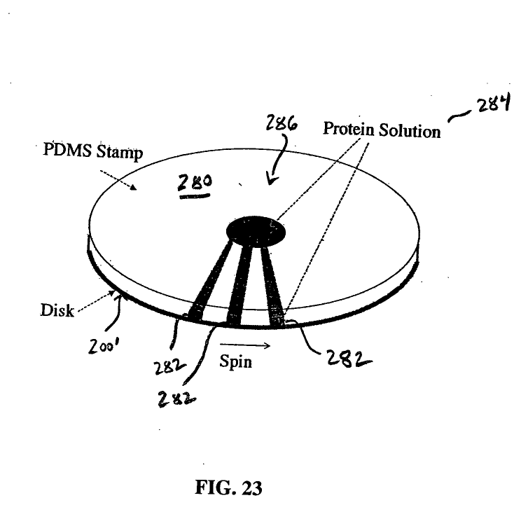

Referring to FIG. 23, an exemplary method for manufacturing CD 200′ is shown. A stamp 280 is used to create a microfluid network including microfluidic channels 282. In one example, stamp 280 is make of PDMS. Stamp 280, similar to stamp 118 is fabricated from a master disk (not shown), similar to glass mold 120. The master disk includes protrusions corresponding to the microfluidic channels 282 of stamp 280. In one example, the protrusions are made from photoresist (SU8-25) patterned onto a flat substrate.

Stamp 280 is next exposed to an oxygen plasma before making contact with CD 200′ to improve the water affinity of the stamp surface of stamp 280 and allow a positive capillary action when a liquid is introduced into channels 282. The surface of CD200′ is configured to bind an analyzer molecule that is to be later introduced to the surface of CD 200′In one example, wherein the surface of CD 200′ is a glass or a silicone the surface is immersed in a solution of chlorodimethy-loctadecylsilane (0.02 M) in an anhydrous toluene solution for at least eight hours. The solution is absorbed by the surface of CD200′ and provides a functional group which attracts the subsequently introduced analyzer molecule. In other examples, different techniques are used to configure CD 200′ to bind the subsequently introduced analyzer molecule.

Stamp 280 is brought into contact with CD 200′ and seals against the surface of CD 200′The sealed system of stamp 280 and CD 200′ is placed on a spinner (not shown) and a solution 284 containing the analyzer molecules is introduced into a central opening 286 of stamp 280 which is in fluid communication with channels 282 of stamp 280. It should be appreciated that stamp 280 can be configured to include multiple openings 286, each opening 286 being in fluid communication with a subset of channels 282. As such, by introducing different solutions 284 to the different openings 286 of stamp 280, CD 200′ may be configured to bind multiple analytes.

Returning to FIG. 23, solution 284 introduced into central opening 286 is communicated to channels 282 and driven towards the outer portions of CD 200′ by capillary force and/or centrifuge force. Solution 284 remains in channels 282 for a sufficient time to permit binding action between CD200′ and the analyzer molecules in solution 284. In one example, solution 284 is in contact with CD200′ for about an hour. Next, channels 282 are rinsed by phosphate-buffered saline solution (PBS) and deionized water under centrifuge force. Stamp 280 is subsequently peeled off CD 200′ and the surface of CD 200′ is blown dry with nitrogen gas. At this point, CD 200′ includes analyzer molecules in regions 270 which correspond to the locations of the channels 282 of stamp 280. CD 200′ is next coated with a blocking material 290 such that regions 272 will not bind the analyte regions 272 are configured to bind.

As stated above, the technology described in U.S. Pat No. 5,900,935, which is incorporated herein by reference, may be adapted to maintain a quadrature condition and its associated increased sensitivity. Instead of a phase modulated signal from an ultrasound source, the present invention so adapted provides a phase modulated signal from analytes present on a CD, such as CD12, CD 100, CD 200.

As described in greater detail in U.S. Pat. No. 5,900,935, a quadrature condition may be maintained by mixing a signal beam from an object under test, such as a ultrasonically vibrated component or a spinning CD, and a reference beam in an adaptive element, such as a real-time holographic element. This quadrature condition is maintained by a phase difference introduced by adaptive holographic element 336 (FIG. 17) and dependent upon the design of holographic element 336, on the applied electric field (E) 337 and on the chosen wavelength for beam 304 from light source 302. Unlike the quadrature condition created due to the height difference between targets 22 and lands 25 of CD 12, system 300 is able to achieve quadrature by adjusting applied electric field 337 or by adjusting the wavelength of laser beam 304. Further, as described in greater detail in U.S. Pat. No. 5,900,935, adaptive element 336 minimizes the effect of low frequency vibrations, such as the wobble of a spinning CD and the effects of laser speckle. Additional details regarding the structure and operation of adaptive element 336 are provided in documents 26 and 29-36.

In one embodiment, adaptive element 336 is a photorefractive multiple quantum well (PRQW). In another embodiment, adaptive element 336 is a photorefractive polymer. In yet another embodiment, adaptive element 336 is a general photorefractive material which exhibits the photorefractive effect. Examples include PRQWs, photorefractive polymers, semiconductors, Barium titanate, lithium niobate, or other suitable photorefractive materials.

Referring initially to FIG. 17, an exemplary adaptive interferometer system 300 including an adaptive element 336 is shown. System 300 includes a laser generator 302 which generates as its output a coherent light beam 304. Light beam 304 is directed in the direction of the adjacent arrow by mirror 306 to beamsplitter 308 which divides beam 304 into a reference beam 320 passing through splitter 308 and a probe beam 324 directed toward the workpiece or material 326 to be examined. Material to be examined 326 is a CD including a biological sample, such as CD 12, CD 100, CD 100′CD 200, and CD 200′ (collectively referred to as “the Bio-CD”). Reference beam 320 is directed by mirror 322 for superposition with the signal wave, as will be described in greater detail below. Probe beam 324 will be reflected or scattered from the Bio-CD as a return signal beam 332 traveling back along its incident path. Tracking control devices such as those described herein are used to align probe beam 324 with a given track of the Bio-CD.

Characteristics of the Bio-CD and turbulence in the optical beam path will cause spatial wavefront distortions on return signal beam 332. Further, the Bio-CD is configured provide a high frequency phase modulation which imports phase perbutations on probe beam 324 when it is reflected back as return signal beam 332. The high frequency phase modulation is created by the spinning of the Bio-CD and the spacing of targets 22 on CD 12, targets 104 and blanks 106 on CD 100, or targets 204 and regions 206 on CD 200. In one example, a given track 102 of CD 100 includes approximately 1,000 targets 104 and CD 100 is spinning at 100 Hz. Thus, the carrier frequency for the high frequency phase modulation is approximately 100 kHz.

The distorted return signal beam 332 is guided toward real-time holographic element 336. Return signal beam 332 is combined or superposed with reference beam 320 in holographic element 336, which results in two output beams 340, 344. The superposition of at least parts of distorted return signal beam 332 and reference beam 320 forms an output beam 340, which is directed to photodetector 346.

The difference in the cumulated path length of beam 320 and the path length of beams 324 and 332 between the beamsplitter 308 and the receiving surface of holographic element 336 should be less than the coherence length of the laser generator 302. In one example, a generally zero path length difference exists between beam 332 and beam 320.

Referring to FIG. 18, the effect of holographic element 336 on the incident beams 320, 332 is shown in greater detail. Reference beam 320 is partially diffracted as beam 320′ and superposed on distorted beam 332 which is partially transmitted as beam 332′. The superposed components of the partially diffracted reference beam 320′ and the partially transmitted signal beam 332′ have identical paths and comprise the resultant beam 340 directed to photodetector 346. The incident reference beam 320 has planar wavefronts 321, while the incident distorted signal beam 332 has distorted wavefronts 333. Resultant beam 340 will have overlapped wavefronts 341 with the same distortion wavefronts 333. Incident reference beam 320 is also partially transmitted through holographic element 336 as component beam 320″, while incident distorted beam 332 is partially diffracted by holographic element 336 as component beam 332″. Component beams 320″, 332″ have identical paths and comprise resultant beam 344. Resultant beam 344 will have overlapped planar wavefronts 345.

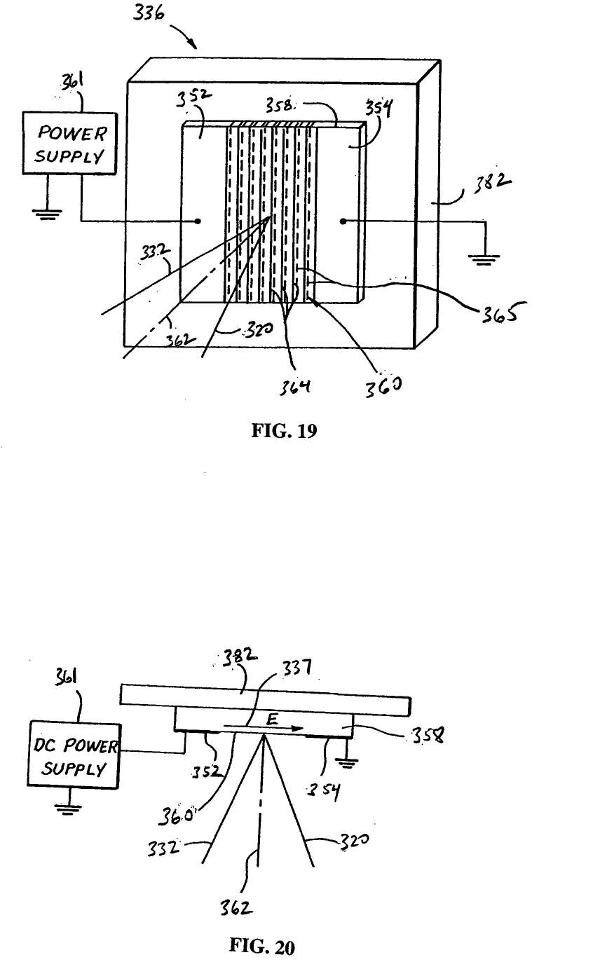

Referring to FIG. 19, a perspective view of the structure of the photorefractive multiple quantum well (PRQW) or holographic adaptive element 336 can be seen in greater detail. Element 336 consists of the semiconductor structure 358 with metal electrodes 352, 354 mounted on a supporting substrate 382 a few millimeters (mm) thick. Substrate 382 may be sapphire, glass or a pyrex material, as is commonly used. Semiconductor structure 358 has a first electrode 352 and a second electrode 354 at opposite ends of the incident surface 360, best seen in FIG. 20, which is a top or plan view of holographic element 336 of FIG. 19. A potential field 337 is maintained across structure 358 between electrodes 352, 354 by a direct current power supply 361. Between electrodes 352, 354, a portion of semiconductor structure 358 is exposed to form incident surface 360. Surface 360 of semiconductor structure 358 receives incident beams 320, 332. A centerline 362 indicating the line normal to surface 360 is also shown.

The incidence of beams 320, 332 onto surface 360 of element 336, referring again to FIGS. 19, 20, results in the intensity grating planes 364, caused by the interfering beams. The intensity grating creates the diffraction grating, shown schematically by the evenly dashed lines 365 in FIG. 19.

In operation, real-time holographic element 336 acts as an adaptive element matching the wavefronts of return signal 332 and reference beam 320. As stated above, return signal 332 acquires a phase perturbation relative to the phase of the reference beam 320 caused by the spinning of the Bio-CD and the repetitive spacing of the associated targets.

When reference beam 320 and return signal beam 332 interfere in the photorefractive multiple quantum well holographic element 336, they produce a complex refractive index and complex grating 365 that records the spatial phase profile of return signal beam 332. This holographic recording and subsequent readout process yields an output beam 340 that is a composite or superposition of the partially transmitted signal beam 332′ and the partially diffracted reference beam 320′ The holographic combination of these beams insures that they have precisely overlapped wavefronts.

The separate beams 320, 332′ that contribute to the composite beam 340 have a static relative longitudinal phase difference apart from the phase perturbation acquired by the return signal 332 from the spinning of the Bio-CD and the repetitive spacing of the associated targets. The static relative longitudinal phase depends on the design of the holographic element 336, on the applied electric field (E) 337 and on the chosen wavelength for beam 304 from light source 302. These factors determine a spatial shift of complex grating 365 in element 336 relative to the optical interference pattern 364 created by return beam 332 and reference beam 320. This spatial shift contributes to the static relative longitudinal phase of the separate beams 320′332′ that contribute to composite beam 340. Specifically, this static relative longitudinal phase is equal to the photorefractive phase shift, plus or minus the wavelength-dependent phase of the signal 320′diffracted by complex grating 365, plus or minus 90 degrees.

Optimally, the static relative longitudinal phase is adjusted in operation such that it is as close as possible to the 90 degree quadrature condition. However, good detection using the principles of this invention is achieved with shifts in the ranges of from 30 degrees to 150 degrees, and from 210 degrees to 330 degrees. In any case, no path-length stabilization is required to maintain this condition as with a conventional interferometer system.

The relative longitudinal phase for the superposed output beam 340 is independent of any wavefront changes on input beams 320, 332 due to turbulence, vibrations, wobble of the Bio-CD, laser speckle, and the like as long as the wavefront changes occur on a time scale that is slow relative to the grating buildup time. The grating buildup time, as used in this specification, is the time required for the amplitude of the refractive index and absorption gratings to reach a given fraction of its final steady-state value. The changes that occur very rapidly, such that the perturbations modulated on the return distorted signal beam 332 as a result of the spinning of the Bio-CD and the spacing of the associated targets will be transferred to the output beam 340 and be detected by the detector 346. It has been found that a suitable detector 346 is Model 1801 provided by New Focus, Inc. of Santa Clara, Calif.

The adaptive holographic element 336, in one embodiment, is able to compensate for mechanical disturbances up to about 10 kHZ or up to about 100 kHz. As such, all disturbances occurring at a rate lower than about 10 kHZ or about 100 kHz will be compensated for by adaptive holographic element 336 while higher frequency signals such as the phase modulation generated by the Bio-CD are passed through adaptive holographic element 336 as a part of output beam 340. For example, assuming the Bio-CD has about 10,000 targets per track and the Bio-CD is rotated at about 6000 revolutions per minute, the sampling rate is approximately 1 MegaSamples/sec, which is above the 100 kHz rate.

As described above, the homodyne interferometer constructed of the photorefractive quantum wells operates by combining two coherent laser beams consisting of signal beam 332 and reference beam 320. Their interference pattern 364 is converted into a complex grating 365. Grating 365 is composed of changes in both the refractive index and the absorption. The periodicity of diffraction grating 365 matches the periodicity of the interference intensity pattern 364 generated by beams 332 and 320. However, complex diffraction grating 365 is generally shifted relative to intensity pattern 364. This spatial shift of the gratings is described in terms of the photorefractive phase shift.

Referring to FIG. 21, an exemplary adaptive interferometer 400 is shown. Adaptive interferometer 400 includes an optical source, laser 402, which emits a beam 404. In one example, laser 402 is a tunable laser diode being tunable from approximately 830 nanometers to about 840 nanometers and available from Melles Griot located at 2051 Palomar Airport Road, 200 Carlsbad, Calif. 92009. Beam 404 passes through a quarter-wave plate 406 and is incident on a polarizing beam splitter 408. Due to the polarization characteristics of beam 404, beamsplitter 408 splits beam 404 into a reference beam 410 and a probe beam 412. The relative intensities of reference beam 410 and probe beam 412 may be adjusted by adjusting quarter-wave plate 406.

Reference beam 410 is redirected by a pair of mirrors 414A, 414B and is finally incident on an adaptive hologram 416. Reference beam 410 also passes through an EO modulator 418 which imparts a phase shift to reference beam 410 and a half-wave plate 420 which alters the polarization state of reference beam 410. EO modulator 418 is optical and is provided to introduce a controlled phase modulation in reference beam 410 for calibrating system 400.

Probe beam 412 passes through a quarter-wave plate 422 which alters the polarization state of probe beam 412 and is focused by a lens 424 onto a track of a spinning Bio-CD. CD 100 is shown for illustration. In one example, lens 424 is a 40x objective lens from a Leica microscope available from Leica Microsystems Inc. located at 2345 Waukegan Road, Bannockburn, IL 60015. Similar tracking control devices described herein are used to align probe beam 412 with a given track of the Bio-CD.

Probe beam 412 is reflected from the analyzer molecule 116 or analyzer molecule/bound analyte on track 102 of CD 100 as a signal beam 430. Signal beam 430 has a wavefront that is altered due to the characteristics of CD 100. The wavefront of signal beam 430 has a periodic phase modulation over time due to the spinning of CD 100 and the repetitive spacing of targets 104 and blanks 106. Signal beam 430 is collected by lens 424 and passes through quarter-wave plate 422 which alters the polarization of signal beam 430 such that the majority of signal beam 430 is transmitted by beamsplitter 408. Signal beam 430, once transmitted by beamsplitter 408, passes through a half-wave plate 432, which alters the polarization of signal beam 430, and is incident on adaptive hologram 416.

Signal beam 430 and reference beam 420 are combined with zero path difference at adaptive hologram 416. Adaptive hologram 416 is generally similar to adaptive hologram 336 described above. In one embodiment, adaptive element 416 is a photorefractive multiple quantum well (PRQW). In another embodiment, adaptive element 416 is a photorefractive polymer. In yet another embodiment, adaptive element 416 is a general photorefractive material which exhibits the photorefractive effect. Examples include PRQWs, photorefractive polymers, semiconductors, Barium titanate, lithium niobate, or other suitable photorefractive materials.

Adaptive hologram 416 generates a diffraction grating (not shown) based on the interference pattern (not shown) of signal beam 430 and reference beam 410. As explained above, adaptive hologram 416 generates two output beams 436 and 438, respectively. Each of output beams 436, 438 passes through a polarizer 440 and is redirected by mirrors 442A, 442B to photodetectors 444A, 444B. Exemplary detectors are Model No. 1801 low-noise amplified photodetectors available from New Focus, Inc. of Santa Clara, Calif.

The signal detected by each of photodetectors 444A, 444B is provided to a lock-in amplifier 446 having a range of about 200 kHz to about 200 MHz and synchronized to the frequency specified by a lock-in amplifier 447 which monitors the track under test on the Bio-CD. It should be noted that the signal from either of photodetectors 444A, 444B may be used to determine the presence or absence of the analyte that track 102 is configured to bind.

The signal from lock-in amplifier 446 is provided to one of an oscilloscope 448 or a processor 450 including software 452 configured to determine based on the signal from lock-in amplifier 446 whether the analyte configured to be bound by the track under test is present or absent.

In one example, the signals from both of photodetectors 444A, 444B are used to determine the presence or absence of the analyte that track 102 is configured to bind. By using both photodetectors 444A, 444B intensity fluctuations can be eliminated because the signal from one of photodetectors 444A, 444B may be subtracted from the signal from the other of photodetectors 444A, 444B to provide a difference signal.

In both systems 300 and 400 the Bio-CD is spun at a given rate between about 1,000 revolutions per minute to about 6,000 revolutions per minute. The respective probe beam 324, 412 is focused on a respective track 24, 102, 202 of the Bio-CD such that as the Bio-CD spins, probe beam 324, 412 sequentially illuminates the respective targets 22, 104, 204 of track 24, 102, 202 and repeats such illumination until system 300, 400 can determine the presence or absence of the analyte configured to be bound to track 24, 102, 202. By rapidly and repeatedly scanning the respective targets 22, 104, 204 of the Bio-CD, system 300, 400 is able to obtain good data averaging with a small detection bandwidth before the probe beam 324, 412 is moved onto the next track 12, 102, 202 of the Bio-CD.

In one exemplary method, wherein CD 100 is used with system 400, a sample potentially containing a first analyte is introduced to CD 100. A first track 102 of CD 100 is probed with system 400, the first track being configured to bind the first analyte. Processor 450 stores at least an indication of the signal received from the first track 102. A control track 102 having similar optical properties as the first track 102 when the first analyte is not present in the sample is probed with system 400. Processor 450 stores at least an indication of the signal received from the control track 102. By comparing the signal received from the first track and the signal received from the control track, processor 450 is able to make a determination whether the analyte is present in the sample or is absent.

In one variation, probe beam 412 of system 400 is split into two probe beams, a first probe beam directed at the first track 102 and a second probe beam directed at the control track 102. Both probe beams are reflected from the respective tracks and are incident on adaptive holographic element 416. Because of the additive properties of holograms, a respective output signal for each of the probe beams may be isolated and monitored with a photodetector. Processor 450 by comparing the output beams based on the first track 102 and the control track 102 is able to determine whether the analyte is present in the sample or absent.

In another exemplary embodiment, wherein CD 200 is used with system 400, a sample potentially containing a first analyte is introduced to CD 200. A first track 202 of CD 200 is probed with system 400, the first track being configured to bind the first analyte. Because of the optical properties of CD 200, namely the generally uniform profile of the specific analyzer molecules 224 and the non-specific antibodies 230, if the first analyte is not present in the sample, then no homodyne signal should occur and processor 450 determines that the first analyte is not present in the sample. However, if the first analyte is present in the sample, then a homodyne signal should be detected and processor 450 determines that the first analyte is present in the sample.

The detection of low-molecular-weight antigens or analytes with system 400 requires maximum sensitivity, which is achieved in the condition of phase-quadrature described above. Below is provided the derivation of a fundamental signal-to-noise ratio for detection in quadrature as a homodyne detection process. For small phase excursions, the total signal is

$$S = \frac{2 {P}_{1}}{hv} m ξ \sqrt{{η}_{p}} \frac{4 π}{λ} Δ {nd}_{An} = \frac{2 {P}_{1}}{hv} m ξ \sqrt{{η}_{p}} \frac{4 π}{λ} Δ {nd}_{An}^{0} {M}_{An} \qquad ( 13 )$$

where P₁ is the signal beam power at the detector, m is the modulation index of the adaptive holographic element 416, ξ is the conversion efficiency from external to internal modulation in the adaptive holographic element 416, ηₚ is the peak diffraction efficiency of the adaptive holographic element 416, Δn is the change in refractive index caused by the bound analyte, and d_{An}=M_{An} d_{An}⁰ is the thickness of the bound analyte layer, where M_{An} are the total number of analyte molecules detected within the detection bandwidth of the experimental system and d_{An}⁰ is the “effective thickness” of a single molecule.

There are three sources of noise in system 300: 1) shot noise of the light; 2) attachment statistics of the antibodies; and 3) bonding statistics of the bound analyte. The shot noise is given by

$${N}_{shot} = \sqrt{\frac{η {P}_{1} BW}{hv}} \qquad ( 14 )$$

where BW is the detection bandwidth of the detection system and η is the detector quantum efficiency. The noise from the fluctuations in the immobilized antibody are given by (assuming random statistics)

$${N}_{An} = 2 \frac{η {P}_{1}}{hv} m ξ \sqrt{{η}_{p}} \frac{4 π}{λ} Δ {n}_{An} {d}_{An}^{0} \sqrt{{M}_{An}} \qquad ( 15 )$$

and for the bound analyte is

$${N}_{Ab} = 2 \frac{η {P}_{1}}{hv} m ξ \sqrt{{η}_{p}} \frac{4 π}{λ} Δ {n}_{Ab} {d}_{Ab}^{0} \sqrt{{M}_{Ab}} \qquad ( 16 )$$

where M _{Ab} and M _{Ab} are the number of bound antibody and analyte molecules, and d_{An}⁰ and d_{Ab}⁰ are the effective thicknesses of a single molecule within the laser spot size.

The total signal-to-noise ratio for the detection is

$$S / N = \frac{\sqrt{{M}_{An}}}{\sqrt{1 + ( \frac{{M}_{Ab}}{{M}_{An}} ) + {( \frac{BW}{\sqrt{{M}_{An} \frac{η {P}_{1}}{hv} Δ {n}_{An} {d}_{An}^{0} 2 m ξ \sqrt{{η}_{p}} \frac{4 π}{λ}}} )}^{2}}} \qquad ( 17 )$$

For ideal operation P₁=P₂, P=P₁+P₂, and m=1. The number of analyte molecules that can be detected when the analyte fluctuation noise equals the shot noise is given be the noise equivalent molecules (NEM).

$$NEM = \frac{1}{\frac{η}{h υ} {( Δ {n}_{An} {d}_{An}^{0} 2 m ξ \sqrt{{η}_{p}} \frac{4 π}{λ} )}^{2}} \qquad ( 18 )$$

for a detected power of 1 Watt and a detection bandwidth of 1 Hz. Assuming Δn=0.1 and d_{An}⁰=0.01 picometer gives a molecular sensitivity of

NEM≈1 molecule Watt per Hz . (19)

With a detector power of about 1 mW and a detection bandwidth of 3 kHz this would place the detection limit at 3 million bound analyte molecules per track, corresponding to 300 molecules per target. As such, a CD having a track for each of the approximately 10,000 blood proteins could detect the presence or absence of each blood protein in a single 10 micro-liter sample without the need for analyte amplification.

For a given system, the shot noise of the laser may be directly measured and the electronic noise of the detector can be characterized. The analyzer molecule noise may be measured by running the Bio-CD without analyte and using a noise mode of the RF lock-in amplifier 446 compared to the noise characteristic of a blank Bio-CD, i.e., a Bio-CD with no analyzer molecules 116. The contribution of the binding analyte to the noise characteristics may be determined through a comparison of an exposed Bio-CD to an unexposed Bio-CD.

Detection systems 300, 400 are both shown with a Bio-CD configured to produce to a reflected signal beam such that detection systems 300, 400 combine the reflected signal beam and a reference beam. Detection systems 300, 400 may be used configured for use with a Bio-CD configured to produce a transmitted signal beam such that detection systems 300, 400 combine the transmitted signal beam and a reference beam.

In one embodiment, the delivery of biological samples containing analytes to the Bio-CD is performed while the Bio-CD is spinning. As stated herein, such delivery can be accomplished using microfluidic channels 56 fabricated in the Bio-CD or by having the biological sample flow over the surface of the Bio-CD. By spinning the Bio-CD centrifugal force pulls the fluid biological sample from the delivery area near the central axis of the Bio-CD outward over the entire surface of the Bio-CD. Further, wherein microfluidic channels are incorporated into the Bio-CD, capillary forces aid in moving the fluid of the biological sample through the microfluidic channels. In alternative embodiments, the biological sample is delivered when the Bio-CD is held stationary.

The delivery of the biological sample while the Bio-CD is spinning further results in an diffusion limited incubation period as opposed to a saturated incubation period. The incubation period is the time period the sample is in contact with the areas of the Bio-CD configured to bind portions of the sample. In one example, the incubation period is on the order of up to several seconds. As a result of the incubation period being diffusion limited, the Bio-CD can be utilized for multiple exposures.

For example, a first biological sample containing the first analyte is introduced to the spinning Bio-CD, the first exposure. During the incubation time the first analyte is bound to approximately 10% of the available analyzer molecules configured to bind the first analyte. The binding of the first analyte during the first exposure is detected by one of the detection systems discussed herein. After the first exposure ninety percent of the of available analyzer molecules configured to bind the first analyte are still capable of binding the first analyte. As such, a second biological sample is introduced to the spinning Bio-CD, the second exposure. Based on the detection of the first analyte corresponding to the first exposure, the detection system can determine if additional first analyte is bound to the Bio-CD during the second exposure and hence present in the second sample.

It should be understood that although the Bio-CD and associated detection systems have been described for use in detecting the presence of blood proteins in a biological sample, the Bio-CD and associated detection systems may be utilized for additional applications such as the analysis of environmental samples including water or other fluidic samples.

While the present system is susceptible to various modifications and alternative forms, exemplary embodiments thereof have been shown by way of example in the drawings and will herein be described in detail. It should be understood, however, that there is no intent to limit the system to the particular forms disclosed, but on the contrary, the intention is to address all modifications, equivalents, and alternatives falling within the spirit and scope of the system as defined by the appended claims.

### TABLE OF REFERENCES

The following table of references includes a plurality of references that are referred to within the disclosure by the corresponding reference number. All of the references listed in the following table of references are expressly incorporated by reference herein.

| Reference Number | Reference |
|---|---|
| 1 | Xia, Y., et al., Non-photolithographic methods for fabrication of |
| | elastomeric stamps for use in microcontact printing. Langmuir, 1996, |
| | Vol. 12, p. 4033-4038. |
| 2 | Hu, J., et al., Using soft lithography to fabricate GaAs/AlGaAs |
| | heterostructue field effect transistors. Appl. Phys. Lett., 1997, Vol. 71, |
| | p. 2020-2022. |
| 3 | Grzybowski, B. A., et al., Generation of micrometer-sized patterns for |
| | microanalytical applications using a laser direct-write method and |
| | microcontact printing. Anal. Chem., 1998, Vol. 70, p. 4645-4652. |
| 4 | Martin, B. D., et al., Direct protein microarray fabrication using a |
| | hydrogel stamper. Langmuir, 1998, Vol. 14, p. 3971-3975. |
| 5 | Pompe, T., et al., submicron contact printing on silicon using stamp |
| | pads. Langmuir, 1999, Vol. 15, p. 2398-2401. |
| 6 | Bietsch, A. and B. Michel, Conformal contact and pattern stability of |
| | stamps used for soft lithography. J. Appl. Phys., 2000, Vol. 88, p. 4310-4318. |
| 7 | Geissler, M., et al., microcontact-printing chemical patterns with flat |
| | stamps. J. Am. Chem. Soc., 2000, Vol. 122, p. 6303-6304. |
| 8 | Sanders, G. H. W. and A. Manz, Chip-based microsystems for genomic |
| | and proteomic analysis. Trends in Anal. Chem., 2000, Vol. 19(6), p. |
| | 364-378. |
| 9 | Wang, J., From DNA biosensors to gene chips. Nucl. Acids Res., 2000, |
| | Vol. 28(16), p. 3011-3016. |
| 10 | Hagman, M., Doing immunology on a chip. Science, 2000, Vol. 290, p. |
| | 82-83. |
| 11 | Marx, J., DNA Arrays reveal cancer in its many forms. Science, 2000, |
| | Vol. 289, p. 1670-1672. |
| 12 | Effenhauser, C. S., et al., Integrated capillary electrophoresis on flexible |
| | silicone microdevices: Analysis of DNA restriction fragments and |
| | detection of single DNA molecules on microchips. Anal. Chem., 1997, |
| | Vol. 69, p. 3451-3457. |
| 13 | He, B. and F. E. Regnier, Anal. Chem., 1998, Vol. 70, p. 3790-3797. |
| 14 | Kricka, L. J., Miniaturization of analytical systems. Clin. Chem., 1998, |
| | Vol. 44(9), p. 2008-2014. |
| 15 | Regnier, F. E., et al., Chromatography and electrophoresis on chips: |
| | critical elements of future integrated, microfluidic analytical systems |
| | for life science. Tibtech, 1999, Vol. 17, p. 101-106. |
| 16 | Ekins, R., F. Chu, and E. Biggart, Development of microspot multi- |
| | analyte ratiometric immunoassay using dual flourescent-labelled |
| | antibodies. Anal. Chim. Acta, 1989, Vol. 227, p. 73-96. |
| 17 | Ekins, R. and F. W. Chu, Multianalyte microspot immunoassay - |
| | Microanalytical “compact Disk” of the future. Clin. Chem., 1991, Vol. |
| | 37(11), p. 1955-1967. |
| 18 | Ekins, R., Ligand assays: from electrophoresis to miniaturized |
| | microarrays. Clin. Chem., 1998, Vol. 44(9), p. 2015-2030. |
| 19 | Gao, H., et al., Immunosensing with photo-immobilized |
| | immunoreagents on planar optical wave guides. Biosensors and |
| | Bioelectronics, 1995, Vol. 10, p. 317-328. |
| 20 | Maisenholder, B., et al., A GaAs/AlGaAs-based refractometer platform |
| | for integrated optical sensing applications. Sensors and Actuators B, |
| | 1997, Vol. 38-39, p. 324-329. |
| 21 | Kunz, R. E., Miniature integrated optical modules for chemical and |
| | biochemical sensing. Sensors and Actuators B, 1997, Vol. 38-39, p. 13-28. |
| 22 | Dubendorfer, J. and R. E. Kunz, Reference pads for miniature |
| | integrated optical sensors. Sensors and Actuators B, 1997 Vol. 38-39, |
| | p. 116-121. |
| 23 | Brecht, A. and G. Gauglitz, recent developments in optical transducers |
| | for chemical or biochemical applications. Sensors and Actuators B, |
| | 1997, Vol. 38-39, p. 1-7. |
| 24 | Hecht, E., Optics. 1987: Addison-Wesely publishing Co., Inc. |
| 25 | Scruby, C. B. and L. E. Drain, Laser Ultrasonics: Techniques and |
| | Applications. 1990, Bristol: Adam Hilger. |
| 26 | Nolte, D. D., et al., Adaptive Beam Combining and Interferometry using |
| | Photorefractive Quantum Wells. J. Opt. Soc. Am. B, Vol. 18, no. 2, |
| | Feb. 2001, pp. 195-205. |
| 27 | John, P. M. S., et al., Diffraction-based cell detection using microcontact |
| | printed antibody grating. Anal. Chem., 1998, Vol. 70, p. 1108-1111. |
| 28 | Morhard, F., et al., Immobilization of antibodies in micropatterns for |
| | cell detection by optical diffraction. Sensors and Actuators B, 2000, |
| | Vol. 70, p. 232-242. |
| 29 | I. Rossomakhin and S. I. Stepanov, “Linear adaptive interferometers via |
| | diffusion recording in cubic photorefractive crystals,” Opt. Commun. |
| | 86, 199-204 (1991). |
| 30 | R. K. Ing and J.-P. Monchalin, “Braodband optical detection of |
| | ultrasound by two-wave mixing in a photorefractive crystal,” Appl. |
| | Phys. Lett. 59, 3233-5 (1991). |
| 31 | Alain Blouin and Jean-Pierre Monchalin, “Detection of ultrasonic |
| | motion of a scattering surface by two-wave mixing in a photorefractive |
| | GaAs crystal,” Appl. Phys. Lett. 65, 932-4 (1994). |
| 32 | B. F. Pouet, R. K. Ing, S. Krishnaswamy, and D. Royer, “Heterodyne |
| | interferometer with two-wave mixing in photorefractive crystals for |
| | ultrasound detection on rough surfaces,” Appl. Phys. Lett. 69, 3782 |
| | (1996). |
| 33 | L-.A Montmorillon, I. Biaggio, P. Delaye, J.-C. Launay, and G. Roosen, |
| | “Eye safe large field of view homodyne detection using a |
| | photorefractive CdTe: V crystal,” Opt. Commun. 129, 293 (1996). |
| 34 | P. Delaye, A. Blouin, D. Drolet, L.-A. Montmorillon, G. Roosen, and |
| | J.-P. Monchalin, “Detection of ultrasonice motion of a scattering |
| | surface by photorefractive InP: Fe under an applied dc field,” J. Opt. |
| | Soc. Am. B14, 1723-34 (1997). |
| 35 | I. Lahiri, L. J. Pyrak-Nolte, D. D. Nolte, M. R. Melloch, R. A. Kruger, |
| | G. D. Bacher, and M. B. Klein, “Laser-Based Ultrasound Detection |
| | using Photorefractive Quantum Wells,” Appl. Phys. Lett. 73, 1041-43 |
| | (1998). |
| 36 | S. Balassubramanian, I. Lahiri, Y. Ding, M. R. Melloch, and D. D. Nolte, |
| | “Two-wave mixing dynamics and nonlinear hot-electorn |
| | transport in transverse-geometry photorefractive quantum wells studies |
| | by moving gratings,” Appl. Phys. B 68, 863-9 (1999). |
| 37 | E. Delamarche, A. Bernard, H. Schmid, B. Michel, and H. Biebuyck, |
| | Science 276, 779-781 (1997). |
| 38 | E. Delamarche, A. Bernard, H. Schmid, A. Bietsch, B. Michel, and H. Biebuyck, |
| | JOURNAL OF THE AMERICAN CHEMICAL SOCIETY |
| | 120, 500-508 (1998). |

What is claimed is:

1. A method for detecting an analyte within a sample, comprising the steps of:
providing a substrate having a plurality of spaced-apart targets configured to bind the analyte;
sequentially directing an optical signal at each of the plurality of spaced-apart targets; and
detecting the presence of the analyte with an interferometer.

2. The method of claim 1, wherein the plurality of spaced-apart targets are arranged in a track and wherein the step of sequentially directing an optical signal at each of the plurality of spaced-apart targets comprises the steps of:
providing an optical source configured to generate the optical signal;
aligning the optical signal with the track; and
spinning the substrate such that the optical signal is sequentially directed at each of the plurality of spaced-apart targets of the track.

3. The method of claim 2, wherein the optical signal is sized to illuminate at least a portion of at least one of the targets and a portion of the substrate surrounding the at least one target.

4. The method of claim 3, wherein the at least one target is at a first height and the portion of the substrate surrounding the target is at a second height that is different from the first height.

5. The method of claim 4, wherein the optical signal generates a first diffraction signal when the analyte is bound to the target and a second diffraction signal when the analyte is not bound to the target, the second diffraction signal being distinguishable from the first diffraction signal.

6. The method of claim 1, wherein the interferometer is an adaptive interferometer.

7. The method of claim 6, wherein the plurality of spaced-apart targets are arranged in a track and wherein the step of sequentially directing an optical signal at each of the plurality of spaced-apart targets comprises the steps of:
providing an optical source configured to generate the optical signal;
aligning the optical signal with the track; and
spinning the substrate such that the optical signal is sequentially directed at each of the plurality of spaced-apart targets of the track.

8. The method of claim 6, wherein the plurality of spaced-apart targets include an antibody configured to bind the analyte.

9. The method of claim 8, wherein the plurality of spaced-apart targets are separated by reference blanks.

10. The method of claim 8, wherein the plurality of spaced-apart targets are separated by a blocking which is configured to not bind the analyte.

11. The method of claim 10, wherein the antibody configured to bind the analyte is stamped onto the substrate.

12. A device for identifying a first analyte of a group of analytes in a biological sample, including:
a substrate including a first plurality of spaced-apart regions configured to bind the first analyte;
an optical source configured to generate a probe beam which illuminates the first plurality of spaced-apart regions in a sequential manner and interacts with the substrate to form a signal beam;
an interferometer configured to combine with an adaptive optical element a reference beam and the signal beam, the combination of the reference beam and the signal beam generating an output beam; and
a detector configured to detect the presence or absence of the first analyte based upon the output beam.

13. The device of claim 12, wherein the probe beam is reflected by the substrate to form the signal beam.

14. The device of claim 12, wherein the probe beam is transmitted through the substrate to form the signal beam.

15. The device of claim 12, wherein each of the first plurality of spaced-apart regions is separated from another of the first plurality of spaced-apart regions by one of a second plurality of regions configured to not bind the first analyte.

16. The device of claim 12, wherein the substrate includes a plurality of concentric tracks spaced such that the probe beam illuminates a single track, the first plurality of spaced-apart regions being disposed on the plurality of concentric tracks.

17. The device of claim 16, further comprising a motor configured to spin the substrate such that the probe beam is sequentially incident on the first plurality of spaced-apart regions of a first track, and a controller configured to control on which track of the plurality of tracks the probe beam is incident.

18. The device of claim 12, wherein the interferometer operates in a quadrature condition.

19. A substrate configured to bind a plurality of analytes and for use with a detection system configured to illuminate the substrate with a probe beam and to determine the presence of one of the analytes on the substrate based on the characteristics of a probe beam subsequent to interacting with the substrate, the substrate comprising:
a circular track including a plurality of spaced-apart targets configured to bind to one analyte, the plurality of spaced-apart targets being separated by a plurality of regions configured to not bind the one analyte; and
wherein the plurality of spaced-apart targets and the plurality of regions together are configured to alter the characteristics of the probe beam to produce a first signal when the one analyte is bound to the plurality of spaced-apart targets and to alter the characteristics of the probe beam to produce a second signal when the one analyte is not bound to the plurality of spaced apart targets.

20. The substrate of claim 19, wherein the plurality of targets of the track each includes a specific analyzer molecule configured to bind the one analyte.

21. The substrate of claim 19, wherein the plurality of targets of the track are at a first height and the plurality of regions of the track are at a second height, the second height being different from the first height.

22. The substrate of claim 21, wherein a height difference between the first height and the second height is approximately one-eighth of a wavelength of the probe beam.

23. The substrate of claim 21, wherein a height difference between the first height and the second height is approximately one-fourth of a wavelength of the probe beam.

24. A device for identifying an analyte in a biological sample, including:
a substrate including a first region configured to bind the analyte upon introduction of the biological sample to the substrate and a second region configured to not bind the analyte;
a motor configured to spin the substrate;
a laser including a beam which is directed at the substrate such that the beam interacts with the first region and the second region as the substrate spins; and
a sensor configured to detect a portion of the beam subsequent to the beam interacting with the substrate, the sensor indicating the presence of the analyte based on an interference characteristic of the beam subsequent to the beam interacting with the substrate, the beam having a first interference characteristic if the analyte is bound to the substrate and a second interference characteristic if the analyte is not bound to the substrate.

25. The device of claim 24 wherein the substrate includes a plurality of first regions and a plurality of second regions.

26. The device of claim 25 wherein the each of plurality of first regions and the plurality of second regions are arranged in an alternating pattern and such that the plurality of first regions and the plurality of second regions form a circular track on the substrate.

27. The device of claim 25 wherein the each of plurality of first regions and the plurality of second regions are arranged in an alternating pattern and each of the plurality of first regions and the plurality of second regions form radially extending spokes on the substrate.

28. The device of claim 27 wherein the plurality of first regions and the plurality of second regions are formed on the substrate by microfluidic printing.

29. The device of claim 24 wherein the substrate includes a plurality of circular concentric tracks, each track including a plurality of first regions and a plurality of second regions, the plurality of first regions and the plurality of second regions being arranged in a repeating pattern.

30. The device of claim 24 wherein the sensor comprises an interferometer configured to combine the portion of the beam subsequent to the beam interacting with the substrate and a reference beam to produce an interference pattern, and a detector configured to provide an indication of the presence or absence of the analyte based at least in part on the interference pattern.

31. The device of claim 30 wherein the portion of the beam subsequent to the beam interacting with the substrate is reflected by the substrate.

32. The device of claim 20 wherein the portion of the beam subsequent to the beam interacting with the substrate is transmitted through the substrate.

33. The device of claim 30 wherein the interferometer includes an adaptive holographic element.

34. The device of claim 24 wherein the beam is generally normal to a surface of the substrate and the first region has a first height and the second region has a second height, the second height being offset relative to the first height.

35. The device of claim 34 wherein the second height is offset relative to the first height by approximately one-eighth of a wavelength of the beam.

36. The device of claim 34 wherein the second height is offset relative to the first height by approximately one-fourth of a wavelength of the beam.

37. A device for identifying analytes in a biological sample, including:
a substrate having a surface lying substantially in a first plane, a plurality of targets offset vertically from the substrate surface, each of the targets having a wall lying substantially in a second plane, one of the surface and the walls of the targets being configured to bind analytes present in the biological sample when the biological sample is introduced to the substrate; and
a laser for sequentially directing a beam at each of the plurality of targets, the laser being positioned relative to the substrate such that when the beam is directed at a target an interference characteristic is produced.

38. The device of claim 37 wherein a first portion of the beam interacts with the target wall and a second portion of the beam interacts with a portion of the substrate adjacent the target, the first and second portions being combined to produce a diffraction signal, the diffraction signal has a first value when an analyte is not bound to the substrate and a second value when an analyte is bound to the substrate, thereby indicating the presence of the analyte.

39. The device of claim 38 wherein the diffraction signal includes the first portion of the beam reflected from the substrate and the second portion of the beam reflected from the substrate.

40. The device of claim 38 wherein the diffraction signal includes the first portion of the beam transmitted through the substrate and the second portion of the beam transmitted through the substrate.

41. A method for detecting the presence of an analyte in a sample, comprising the steps of:
providing a substrate having a plurality of first regions configured to bind the analyte and a plurality of second regions configured to not bind the analyte;
spinning the substrate;
introducing the sample proximate the center of the substrate and continuing to spin the substrate such that an incubation time for the sample is generally diffusion limited;
directing a laser beam at the substrate such that the laser interacts with the substrate; and
determining the presence of the analyte based at least in part on a characteristics of laser beam subsequent to interaction with the substrate.

42. The method of claim 41 wherein the step of determining the presence of the analyte based at least in part on a characteristics of the portion of the laser beam subsequent to interaction with the substrate includes of the steps of
combining the portion of the laser beam subsequent to interaction with the substrate with a reference beam, such that the portion of the laser beam subsequent to interaction with the substrate and the reference beam create an interference pattern;
locating an adaptive element such that the interference pattern interacts with the adaptive element, the adaptive element generating at least a first output signal; and
detecting the first output signal.

43. The method of claim 41 wherein the plurality of first regions are formed on the substrate by microfluidic printing.

44. The method of claim 41 wherein the plurality of first regions are formed on the substrate by ink jet printing.
